(19) United States
(12) Patent Application Publication
(10) Pub. No.: US 2016/0237496 A1
(43) Pub. Date: Aug. 18, 2016

(54) METHODS FOR ASSESSING STATUS OF POST-TRANSPLANT LIVER AND DETERMINING AND ADMINISTERING SPECIFIC TREATMENT REGIMENS

(71) Applicants: HITACHICHEMICAL COMPANY LTD., Chiyoda-ku, Tokyo (JP); HITACHI CHEMICAL CO. AMERICA, LTD., Cupertino, CA (US); NATIONAL UNIVERSITY CORPORATION KUMAMOTO UNIVERSITY, Kumamoto (JP)

(72) Inventors: Masato Mitsuhashi, Irvine, CA (US); Daiki YOSHII, Kumamato-Shi (JP); Katsuhiro ASONUMA, Kumamoto-Shi (JP); Yukihiro INOMATA, Kumamoto-Shi (JP)

(21) Appl. No.: 15/025,209

(22) PCT Filed: Sep. 30, 2014
(86) PCT No.: PCT/US14/58404

§ 371 Date: Mar. 25, 2016

Related U.S. Application Data
(60) Provisional application No. 61/885,755, filed on Oct. 2, 2013.

(51) Int. Cl. C12Q 1/68

(52) U.S. Cl. CPC C12Q 1/6883; C12Q 1/6806; C12Q 2600/158

(57) ABSTRACT

Methods and devices allow assessment of the status of a transplanted liver during the post-transplant period. The methods are particularly beneficial for identifying if a transplanted liver is subject to rejection, by what mechanisms, and thereby developing and implementing a specific treatment regime to reduce the rejection of the transplanted liver.

### BACKGROUND

1. Field of the Invention

The present disclosure relates generally to methods for assessing the status of a transplanted liver in a recipient in order to determine the presence or absence of rejection of transplanted liver. In particular, in several embodiments methods are disclosed for generating and implementing a specifically designed treatment regime to resolve rejection of the transplanted liver, based specifically on an individual patient's rejection symptoms.

2. Description of Related Art

Organ transplantation, moving an organ from a donor site to a recipient site (either from a first to a second subject or from a first to a second location on a patient's own body) has been practiced in medicine for many centuries, with the first documented successful transplants occurring regularly in the early 1900's. Transplants are performed in order to replace the recipient's damaged or absent organ. While more recently, regenerative medicine and cell therapy has been focused on use of cells to treat damaged or diseased tissues, transplant of entire organs is still commonplace.

Worldwide, the kidneys are the most commonly transplanted organs, followed closely by the liver and then the heart. The cornea and bones and/or tendons (e.g., musculoskeletal grafts) are the most commonly transplanted tissues, transplanted tenfold more than whole organs. Other transplants include lungs, pancreas, intestine, thymus, skin, heart valves, and veins. While generally controllable, transplants still pose the risk of rejection of the transplanted organ or tissue by the recipient. Diagnosis of rejection is typically via study of clinical data, such as patient signs and symptoms, as well as laboratory data such as tissue biopsy.

### SUMMARY

Despite the advances in organ transplantation, there remains the possibility of infection of the transplanted organ and/or rejection of the organ by the recipient. Improvement in the long-term success of organ transplants, can be facilitated by early detection and treatment of infection or rejection, which are described herein. In several embodiments, there are provided methods of treating the liver of a subject, comprising obtaining a biological sample from the liver (such as bile), ordering a test of the (sample (e.g., bile), obtaining the results of the test, evaluating the results to determine the status (e.g., health and/or function) of the liver of the subject. In several embodiments, the test is configured to identify the status of the liver of the subject as other than being in a recovery phase from acute rejection. In some embodiments, the test comprises isolating components from the sample (e.g., bile), liberating RNA from the isolated components, contacting the liberated RNA with a reverse transcriptase to generate complementary DNA (cDNA), and contacting the cDNA with sense and antisense primers that are specific for markers of liver condition and a DNA polymerase in order to generate amplified DNA, followed by detecting the amount of expression of the markers of liver condition.

In some embodiments, there is provided a method of determining the status of a liver of a subject, comprising: obtaining bile collected from the liver, isolating one or more biological components (e.g., vesicles, exosomes, microvesicles, or the like) from the bile by passing the bile through a filter (e.g., a membrane or plurality of membranes) configured to capture one or more of the biological components, detecting expression of at least one marker of liver condition, and identifying the status of the liver of the subject. In some embodiments, the detecting step further comprises the steps of isolating RNA from the collected bile, contacting RNA from the collected bile with a reverse transcriptase in order to generate complementary DNA (cDNA), and contacting the cDNA with sense and antisense primers that are specific for one of the markers of liver condition and a DNA polymerase to generate amplified DNA.

In other embodiments a method of determining the status of a liver comprises: obtaining bile collected from the liver, isolating one or more biological component from the bile by passing the bile through a membrane configured to capture one or more of the biological components, detecting expression of at least one marker of liver condition, and identifying status of the liver of the subject. In some embodiments, the detecting step further comprises the steps of isolating RNA from the collected bile, contacting RNA from the collected bile with a reverse transcriptase to generation complementary DNA (cDNA), and contacting said cDNA with sense and antisense primers that are specific for the marker of liver condition.

In some embodiments, a method of directing treatment of the liver of a subject is provided that comprises: receiving bile collected from the liver of the subject, detecting expression of at least one marker of liver condition, identifying the status of the liver of the subject, and informing a physician that it would be appropriate to treat the subject if the subject as indicated by the identified status of the liver. In some embodiments, detecting expression is performed by a method comprising: isolating one or more biological components from the bile, liberating RNA from the isolated biological components, contacting the liberated RNA with a reverse transcriptase to generate complementary DNA (cDNA), and contacting the cDNA with sense and antisense primers that are specific for each of the markers of liver condition and a DNA polymerase in order to generate amplified DNA.

In some embodiments, there is provided a method of identifying the status of a liver of a subject after a liver transplant, comprising: obtaining bile collected from the liver, isolating one or more biological components from the bile, liberating RNA from the isolated biological components, contacting the liberated RNA with a reverse transcriptase to generate complementary DNA (cDNA), detecting expression of at least one marker of liver condition by a computerized method, and using a computer to identify the status of the liver of the subject. In some embodiments, detecting expression by a computerized method comprises the steps of: contacting the cDNA with sense and antisense primers that are specific for each of the markers of liver condition and a DNA polymerase to generate a reaction mixture; exposing the reaction mixture to a thermal cycle.

In some embodiments, a method of identifying the status of a liver of a subject is provided that comprises: obtaining bile collected from the liver of the subject, isolating one or more biological components from the bile, detecting expression of at least one marker of liver condition, and identifying the status of the liver of the subject. In some embodiments, the detecting expression step is performed by a method comprising: liberating RNA from the isolated membrane particles, exosomes, exosome-like vesicles, and/or microvesicles; contacting the liberated RNA with a reverse transcriptase to generate complementary DNA (cDNA); and contacting the cDNA with sense and antisense primers that are specific for each of the markers of liver condition and a DNA polymerase in order to generate amplified DNA.

In some embodiments, the methods comprise collection of bile (or another liver-associated biological sample) from the liver of the subject after a liver transplant. In some embodiments, the methods herein are performed and bile is collected form the liver of the subject after liver surgery. In other embodiments, bile is collected prior to a liver transplant or liver surgery. In some embodiments, the methods herein may be performed by collected bile from an individual with a healthy liver. In other embodiments, the individual's liver may be diseased or otherwise unhealthy. In some instance, the methods described herein may be performed on a foreign transplanted liver.

In some embodiments, components isolated from the bile are one or more of membrane particles, exosomes, exosome-like vesicles, and microvesicles. In other embodiments, components isolated from the bile may be any biological component that comprises RNA or DNA.

In some embodiments, isolating the biological components of interest from the bile comprises filtering the bile. In some embodiments, filtering the bile will trap one or more of membrane particles, exosomes, exosome-like vesicles, and microvesicles on a filter. In some embodiments, the filter comprises material to capture components that are about 1.6 microns or greater in diameter. In several embodiments, a plurality of filters are used to capture vesicles within a particularly preferred range of sizes (e.g., diameters). For example, in several embodiments, filters are used to capture vesicles having a diameter of from about 0.2 microns to about 1.6 microns in diameter, including about 0.2 microns to about 0.4 microns, about 0.4 microns to about 0.6 microns, about 0.6 microns to about 0.8 microns, about 0.8 microns to about 1.0 microns, about 1.0 microns to about 1.2 microns, about 1.2 to about 1.4 microns, about 1.4 microns to about 1.6 microns (and any size in between those listed).

In some embodiments, the filter (or filters) comprises glass-like material, non-glass-like material, or a combination thereof. In some embodiments, the bile is passed through multiple filters to isolate the biological component of interest. In other embodiments, isolating biological components comprises diluting the bile. In other embodiments, centrifugation may be used to isolate the biological components of interest. In some embodiments, multiple isolation techniques may be employed (e.g., combinations of filtration selection and/or density centrifugation). In some embodiments, the bile is separated into one or more samples after the isolating step.

In some embodiments, a filter device is used to isolate biological components of interest. In some embodiments, the device comprises: a first body having an inlet, an outlet, and an interior volume between the inlet and the outlet; a second body having an inlet, an outlet, an interior volume between the inlet and the outlet, a filter material positioned within the interior volume of the second body and in fluid communication with the first body; and a receiving vessel having an inlet, a closed end opposite the inlet and interior cavity. In some embodiments, the first body and the second body are reversibly connected by an interaction of the inlet of the second body with the outlet of the first body. In some embodiments, the interior cavity of the receiving vessel is dimensioned to reversibly enclose both the first and the second body and to receive bile after it is passed from the interior volume of the first body, through the filter material, through the interior cavity of the second body and out of the outlet of the second body. In some embodiments, the isolating step comprises placing at least a portion of the bile in such a device, and applying a force to the device to cause bile to pass through the device to the receiving vessel and capture the biological component of interest. In some embodiments, applying the force comprises centrifugation of the device. In other embodiments, applying the force comprises application of positive pressure to the device. In other embodiments, applying the force comprises application of vacuum pressure to the device.

In some embodiments, liberating the RNA from the biological component of interest comprises lysing the membrane particles, exosomes, exosome-like vesicles, and/or microvesicles with a lysis buffer. In other embodiments, centrifugation may be employed. In some embodiments, the liberating is performed while the membrane particles, exosomes, exosome-like vesicles, microvesicles and/or other components of interest are immobilized on a filter. In some embodiments, the membrane particles, exosomes, exosome-like vesicles, microvesicles and/or other components of interest are isolated or otherwise separated from other components of the bile (and/or from one another—e.g., vesicles separated from exosomes).

In some embodiments, the chosen markers of liver condition indicate that a liver is healthy. In other embodiments, the chosen markers of liver condition indicate that a liver is unhealthy or diseased (e.g., as compared to a prior evaluation of the liver of a particular subject, or as compared to the general population/accepted clinical norms), or that a transplanted liver is being rejected. For example, certain markers of liver condition may indicate that a transplanted liver is in an early stage of acute rejection or early stage of acute infection; in acute rejection or acute infection; in sustained rejection or sustained infection; or in recovery.

In some embodiments, certain families of markers may indicate the status of the liver. For example the status of a liver may be determined as in an early stage of acute rejection or early stage of acute infection when one or more of macrophage-derived mRNAs, IL8, and chemokine mRNAs is detected. In some embodiments, the status of a liver may be determined as in acute rejection or acute infection when one or more of cytotoxic T-cell derived mRNAs (TNF⁻ FasL, IFNG, GZB), or leukocyte-specific mRNAs (CD16, DEFA3) is detected. In additional embodiments, the status of a liver may be determined as in a recovery phase from acute rejection or acute infection when one or more of regulatory T-cell derived or anti-inflammatory cytokine mRNAs (IL10, TGFB, CTLA4, PD-1, FOXP3 is detected. In some embodiments, the status of a liver may be determined to be in sustained rejection or sustained infection when one or more of Th1-(IL2), Th2-(IL4) derived mRNAs or GMCSF is detected. In some embodiments, the methods herein are performed by selecting a marker from each of these families of markers and detecting the expression of each of the selected markers in order to determine the status of the liver.

In some embodiments, markers are selected from the group consisting of IL1B, IL6, IL8, TNF-alpha, FasL, IFNG, granzyme B, CD16, DEFA3, IL10, TGF beta, CTLA4, PD-1, FOXP3, IL2, IL4 and GMCSF. In some embodiments the status of a liver may be determined as in an early stage of acute rejection or early stage of acute infection when one or more of IL1B, IL6, and IL8 is detected. In some embodiments, the status of a liver may be determined as in acute rejection or acute infection when one or more of TNF-alpha, FasL, IFNG, granzyme B, CD16, and DEFA3 is detected. In some embodiments, the status of a liver may be determined as in a recovery phase from acute rejection when one or more of IL10, TGF-beta, CTLA4, PD-1 and FOXP3 is detected. In some embodiments, the status of a liver may be determined as in sustained rejection or sustained infection when one or more of IL2, IL4 and GMCSF is detected. In some embodiments, the methods herein are performed by selecting a marker from each of these families of markers and detecting the expression of each of the selected markers in order to determine the status of the liver. In some embodiments, none of

In some embodiments, the methods further comprise informing a physician that treatment of the subject is appropriate if the subject is not in the recovery phase from acute rejection. In some embodiments, the physician is advised to treat the subject when none of the markers of the recovery phase of acute rejection or infection, including IL10, TGF-beta, CTLA4, PD-1 or FOXP3, are detected. In some embodiments, IL1B, IL6, or IL8 is detected and rejection is not considered to be clinically relevant. In some embodiments, a physician is advised not to treat a subject when rejection is not considered to be clinically relevant. However, in other instances, both markers of the recovery phase of acute rejection or infection and markers of another form of rejection or infection may be detected. In some embodiments, advising a physician to treat the subject may be appropriate based on these results. In some embodiments, IL1B, IL6, or IL8 is detected and rejection is not considered to be clinically relevant. In some embodiments, a subject is treated with administration of antibiotic therapy (alone or in combination with other immune-boosting therapy) in response to this scenario. In some embodiments, both markers of early stage of acute rejection or early stage of acute infection and markers of acute rejection or acute infection are detected. In other embodiments, both markers of early stage of acute rejection or early stage of acute infection and markers of sustained rejection or sustained infection are detected. In other embodiments, both markers of acute rejection or acute infection and markers of sustained rejection or sustained infection are detected. In some embodiments, markers of early rejection or infection, acute rejection or infection, and sustained rejection or infection are all detected. In several such embodiments, additional tests are used to further determine if and how a patient should be treated. However, in some embodiments, the absolute change of one category of markers as compared to another (optionally normalized to a control) allow a determination of if and how a patient should be treated. For example, if the change in expression (e.g., an increase) of markers of sustained infection is greater than those for acute infection, a medical provider may deem it appropriate to treat the subject for sustained infection. In some embodiments, a plurality of samples is taken over time, so that a determination can be made as to whether a subject is progressing from a state of acute to sustained infection or, alternatively from a state of sustained infection to a healthier state.

In some embodiments, treating comprises a treatment selected from the group of removal of transplanted tissue, re-transplant, immunosuppressive therapy, antibody-based or antibiotic treatments, blood transfusions, or bone marrow transplant.

In some embodiments, the RNA liberated from the biological components of interest comprises poly(A)+ RNA.

In some embodiments, after amplified DNA is generated, it is exposed to a probe complementary to a portion of one of the markers of liver condition.

In some embodiments, the test of the bile or the identified liver status is corroborated with a histological evaluation of a biopsy of the liver. In other embodiments, the test of the bile or the identified liver status further comprises comparing the expression of the markers of liver condition in the subject to the expression of the markers of liver condition in a control sample.

In some embodiments, a computerized method is used to complete one or more of the steps. In some embodiments, the computerized method comprises exposing a reaction mixture comprising isolated RNA and/or prepared cDNA, a polymerase and gene-specific primers to a thermal cycle. In some embodiments, the thermal cycle is generated by a computer configured to control the temperature time, and cycle number to which the reaction mixture is exposed. In other embodiments, the computer controls only the time or only the temperature for the reaction mixture and an individual controls on or more additional variables. In some embodiments, a computer is used that is configured to receive data from the detecting step and to implement a program that detects the number of thermal cycles required for the marker of liver condition to reach a pre-defined amplification threshold in order to identify the status of the liver. In still additional embodiments, the entire testing and detection process is automated.

For example, in some embodiments, RNA is isolated by a fully automated method, e.g., methods controlled by a computer processor and associated automated machinery. In one embodiment a biological sample, such as a bile sample, is collected and loaded into a receiving vessel that is placed into a sample processing unit. A user enters information into a data input receiver, such information related to sample identity, the sample quantity, and/or specific patient characteristics. The user can then implement an RNA isolation protocol, for which the computer is configured to access an algorithm and perform associated functions to process the bile sample in order to isolate biological components, such as vesicles, and subsequently processed the vesicles to liberate RNA. In further embodiments, the computer implemented program can quantify the amount of RNA isolated and/or evaluate and purity. In such embodiments, should the quantity and/or purity surpass a minimum threshold, the RNA can be further processed, in an automated fashion, to generate complementary DNA (cDNA). cDNna can then be generated using established methods, such as for example, binding of a poly-A RNA tail to an oligo dT molecule and subsequent extension using an RNA polymerase.

Depending on the embodiment, the cDNA can be divided into individual subsamples, some being stored for later analysis and some being analyzed immediately. Analysis, in some embodiments comprises mixing a known quantity of the cDNA with a salt-based buffer, a DNA polymerase, and at least one gene specific primer to generate a reaction mixture. The cDNA can then be amplified using a predetermined thermal cycle program that the computer system is configured to implement. This thermal cycle, could optionally be controlled manually as well. After amplification (e.g., real-time PCR,), the computer system can assess the number of cycles required for a gene of interest (e.g. a marker of liver specific function) to surpass a particular threshold of expression. A data analysis processor can then use this assessment to calculate the amount of the gene of interest present in the original sample, and by comparison either to a different patient sample, a known control, or a combination thereof, expression level of the gene of interest can be calculated. A data output processor can provide this information, either electronically in another acceptable format, to a test facility and/or directly to a medical care provider. Based on this determination, the medical care provider can then determine if and how to treat a particular patient based on the assessment of the status of the liver post-transplant.

In several embodiments, there are provided methods for determining the status (e.g., the level of function and/or health) of a liver of subject. In several embodiments, the status is determined shortly after a liver transplant, or a liver surgery (or other treatment). For example, in several embodiments, there is provided a method of identifying the status of a liver of a subject after a liver transplant, comprising (I) obtaining bile collected the liver of the subject after the liver transplant, (II) isolating one or more of membrane particles, exosomes, exosome-like vesicles, and microvesicles from said bile, (III) detecting expression of at least one marker of liver condition from each of the following groups of markers: (a) IL1B, IL6 and IL8, (b) TNF-alpha, FasL, IFNG, granzyme B, CD16, DEFA3, and PRG2, (c) IL10, TGF beta, CTLA4, PD-1 and FOXP3, and (d) IL2, IL4 and GMCSF by a method comprising (i) liberating RNA from the isolated membrane particles, exosomes, exosome-like vesicles, and/or microvesicles, (ii) contacting the liberated RNA with a reverse transcriptase to generate complementary DNA (cDNA), and (iii) contacting said cDNA with sense and antisense primers that are specific for each of the markers of liver condition and a DNA polymerase in order to generate amplified DNA; and (IV) identifying status of the liver of the subject as (a) in an early stage of acute rejection or early stage of acute infection when one or more of IL1B, IL6, and IL8 is detected, (b) in acute rejection when one or more of TNF-alpha, FasL, IFNG, granzyme B, CD16, and DEFA3 is detected, (c) in a recovery phase from acute rejection when one or more of IL10, TGF-beta, CTLA4, PD-1 and FOXP3 is detected, or (d) in sustained rejection when one or more of IL2, IL4 and GMCSF is detected.

In several embodiments, there is provided a method of determining the status of a liver of a subject after a liver transplant, comprising obtaining bile collected from the liver, isolating one or more of membrane particles, exosomes, exosome-like vesicles, and microvesicles from said bile by passing said bile through a membrane configured to capture one or more of said membrane particles, exosomes, exosome-like vesicles, and microvesicles, detecting expression of at least one marker of liver condition selected from the group consisting of IL1B, IL6, IL8, TNF-alpha, FasL, IFNG, granzyme B, CD16, DEFA3, IL10, TGF beta, CTLA4, PD-1, FOXP3, IL2, IL4 and GMCSF by a method comprising, (i) isolating RNA from the collected bile, (ii) contacting RNA from the collected bile with a reverse transcriptase in order to generate complementary DNA (cDNA), and (iii) contacting said cDNA with sense and antisense primers that are specific for one of IL1B, IL6, IL8, TNF-alpha, FasL, IFNG, granzyme B, CD16, DEFA3, IL10, TGF beta, CTLA4, PD-1, FOXP3, IL2, IL4 and GMCSF and a DNA polymerase to generate amplified DNA, and identifying status of the liver of the subject as: (a) in an early stage of acute rejection or early stage of acute infection when one or more of IL1B, IL6, and IL8 is detected, (b) in acute rejection when one or more of TNF-alpha, FasL, IFNG, granzyme B, CD16, and DEFA3 is detected, (c) in a recovery phase from acute rejection when one or more of IL10, TGF-beta, CTLA4, PD-1 and FOXP3 is detected, or (d) in sustained rejection when one or more of IL2, IL4 and GMCSF is detected.

Additionally, there are provided methods of determining the status of a liver of a subject after a liver surgery, comprising: obtaining bile collected from the liver, isolating one or more of membrane particles, exosomes, exosome-like vesicles, and microvesicles from said bile by passing said bile through a membrane configured to capture one or more of said membrane particles, exosomes, exosome-like vesicles, and microvesicles, detecting expression of at least one marker of liver condition by a method comprising: (i) isolating RNA from the collected bile; (ii) contacting RNA from the collected bile with a reverse transcriptase to generation complementary DNA (cDNA); and (iii) contacting said cDNA with sense and antisense primers that are specific for said marker of liver condition, and identifying status of the liver of the subject as: (a) in an early stage of acute rejection or early stage of acute infection when one or more of macrophage-derived mRNAs, IL8, and chemokine mRNAs is detected, (b) in acute rejection when one or more of cytotoxic T-cell derived mRNAs (TNF alpha, FasL, IFNG, GZB), or leukocyte-specific mRNAs (CD16, DEFA3) is detected, (c) in a recovery phase from acute rejection when one or more of when regulatory T-cell derived or anti-inflammatory cytokine mRNAs (IL10, TGFB, CTLA4, PD-1, FOXP3 is detected, or (d) in sustained rejection when one or more of Th1-(IL2), Th2-(IL4) derived mRNAs or GMCSF is detected. [0007] In several embodiments, the isolating comprises filtering said bile. In some embodiments, the isolating comprises diluting and filtering said bile. In several embodiments, the filtration traps one or more of membrane particles, exosomes, exosome-like vesicles, and microvesicles on the filter. In this manner, several samples of bile can be processed sequentially, if there is a dilute vesicular concentration in each of the samples. Advantageously, this allows for a many-fold concentration of captured membrane particles. However, in several embodiments, a single sample allows for capture of a sufficient amount of membrane particles to enable a full analysis of the status of the subject's liver.

Additionally, in several embodiments, the isolating comprises placing at least a portion of said bile into a device comprising a first body having an inlet, an outlet, and an interior volume between the inlet and the outlet, a second body having an inlet, an outlet, an interior volume between the inlet and the outlet, a filter material positioned within the interior volume of the second body, and in fluid communication with said first body, wherein the first body and the second body are reversibly connected by an interaction of the inlet of the second body with the outlet of the first body, and a receiving vessel having an inlet, a closed end opposite the inlet and interior cavity, wherein the interior cavity of the receiving vessel is dimensioned to reversibly enclose both the first and the second body and to receive the bile after it is passed from the interior volume of the first body, through the filter material, through the interior cavity of the second body and out of the outlet of the second body; and centrifuging said device to cause said bile to pass through the device to the receiving vessel and capture said membrane particles, exosomes, exosome-like vesicles, and/or microvesicles on said filter material.

In several embodiments, the liberating comprises lysing said membrane particles, exosomes, exosome-like vesicles, and/or microvesicles. In some embodiments, the lysing is performed while said membrane particles, exosomes, exosome-like vesicles, and/or microvesicles are trapped on said filter.

In several embodiments, the RNA comprises poly(A)+ RNA. In several embodiments, the generated amplified DNA is exposed to a probe complementary to a portion of one of said markers of liver condition.

As an optional step, several embodiments, further comprise corroborating the identified liver status with histological evaluation of a biopsy of said liver. Moreover, in several embodiments, the methods further comprise treating the patient according to the outcome of the methods. For example, in several embodiments, the subject is treated according to whether the status of the subject's liver is identified as in a stage of early acute rejection, in acute rejection, in a recover phase, or in sustained rejection. Thus, in several embodiments, not only can the status of the subject's be determined in a timely and accurate fashion, but an appropriate treatment regimen can be prepared and/or implemented.

The methods summarized above and set forth in further detail below describe certain actions taken by a practitioner; however, it should be understood that they can also include the instruction of those actions by another party. Thus, actions such as “administering a treatment to a subject after determining the subject is suffering from liver transplant rejection” include “instructing the administration of a treatment to a subject after determining the subject is suffering from liver transplant rejection.”

### BRIEF DESCRIPTION OF THE FIGURES

FIG. 1 depicts a process flow scheme used to capture exosomes (or other membrane-bound bodies) and process the nucleic acids in a sample in order to assess gene expression.

FIG. 2 is a cross-section view of one embodiment of a capture device as disclosed herein.

FIG. 3 is a cross-section view of one embodiment of a first hollow body as disclosed herein.

FIG. 4 is a cross-section view of one embodiment of a second hollow body as disclosed herein.

FIG. 5 is a cross-section view of an additional embodiment of a second hollow body as disclosed herein.

FIG. 6 is a cross-section view of microvesicle capture system as disclosed herein.

FIG. 7 depicts a timeline of rejection and recovery in a liver transplant subject and various diagnostic parameters that were evaluated.

FIGS. 8A-8B depicts hematoxylin and eosin staining of liver tissue sections that demonstrate infiltration of immune cells, indicative of transplant rejection.

FIGS. 9A-9Q depict gene expression analysis of a variety of markers of immune function that were isolated from bile samples from post-transplant patients.

### DETAILED DESCRIPTION

With nearly 120,000 men, women, and children awaiting organ or tissue transplants in the United States, some of them requiring a second (or greater) transplant, the need for successful long-term transplants is paramount. As transplants have become much more common in the last several decades, preparation and post-transplant medical care has become more sophisticated and led to improved transplant success rates.

Transplants, however, still run the risk of various clinical problems, such as rejection, infection, relapse of original disease, drug toxicity, etc. Early diagnosis and differential diagnosis are critically important for the timing of treatment as well as the choice of appropriate drugs or drug combinations. Although various clinical parameters are available to initially identify rejection, such as, for example, fever, leukocytosis, elevation of CRP, serum biochemical parameters, etc., these are not specific enough to identify the nature of the problem. Biopsy is frequently carried out for definite diagnosis, but it is invasive and is not applicable routinely. Thus, there is a clear demand for better diagnostic procedures and treatment methods, which are provided in several embodiments of the methods disclosed herein.

### Transplant and Rejection

Organ and tissue transplant, while not mainstream in the medical field, has become much more prominent as techniques for controlling complications with respect to transplant surgery, or post-surgery, have improved. Transplants may consist of transplantation of organs (e.g., an entire organ). Alternatively, transplants may comprise transplantation of a tissue, in other words a portion of an organ such as, for example, muscle, tendon, connective tissue, or skin. Organs that are transplanted include, but are not limited to, kidney, heart, liver, lungs etc. Tissues that are transplanted include, but are not limited to, muscle, tendon, connective tissue, skin, eyes, and/or cells. Depending on the embodiment, transplantation may be in one of several forms. For example tissue transplantation is often autologous (donor and recipient are same individual). Organ transplantation, on the other hand, is often allogeneic (donor and recipient are different individuals). However, depending on the embodiment disclosed herein, transplantation of organs or tissues may be autologous or allogeneic. In additional embodiments, xenogeneic transplants occur. Instill additional embodiments, syngeneic transplants occur. In some embodiments the methods disclosed herein are used to evaluate the post-transplants status of an individual having received an ABO incompatible transplant. Such transplants enable the use of organs for donation regardless of AB blood type, though they are typically limited to infant recipients. Regardless of the type of transplant has occurred, the methods disclosed herein are useful for assessing the condition of the recipient post-transplant and identifying (i) the presence of transplant rejection, (ii) the source or sources of the rejection, and (iii) the likely most efficacious treatment regime to address the rejection.

A variety of different mechanisms may come into play post-transplant that lead to rejection of a transplanted organ by the recipient. Organ or tissue rejection is an immune response that involves both the cellular immune pathway and humoral immune pathways. Cellular immunity is mediated by killer T cells which induce apoptosis of target cells, in this case, the cells of the transplanted organ. Humoral immunity is mediated by activated B cells that secrete antibody molecules that are directed against the transplanted tissue. In some cases rejection also involves the innate immune response. Depending on the type of transplant (e.g., the tissue involved), various rejection mechanisms may come into play. However, advantageously, the methods disclosed herein allow evaluation of the post-transplant status of the transplanted organ or tissue based on, for example, analysis of expression of organ or tissue-function related markers, such as, for example mRNA.

Post-transplant, donor dendritic cells (the primary antigen presenting cells) are released from the donor tissue or organ and move to and present in the recipient's lymphoid tissue (such as their lymph nodes). In this presentation, the dendritic cells present the donors “self peptides” to the recipient lymphocytes these lymphocytes (e.g. T cells, such as helper T cells and/or killer T cells, and B cells) enact specific immunity. This specific immunity then results in the immune responses that are directed specifically at the donor “self peptides”, thus raising immune responses to, and eventually rejection of, the donated tissue or organ.

Cellular immunity is a result of killer T cells (also known as cytotoxic T lymphocytes) having CD8 surface receptors that interact with the major histocompatibility complex class I molecules on transplant tissue from a donor. The MHC class I molecules display the donor's “self peptides”. After this interaction with the with the MHC class I molecules of the transplanted tissue (via the T cell receptor a.k.a. TCR) the killer T cells can then recognize their matching epitopes and trigger apoptosis of that target cell (or cells) thereby resulting in a reduced function or complete rejection of the transplanted organ or tissue.

Often times, a transplant recipient may have been exposed previously to an antigen that leads to specific immunity. This can occur for example if there were a blood type mismatch during a previous blood transfusion, such as a transfusion during the organ transplantation. Upon a subsequent exposure to the foreign antigens, pre-existing cross-reactive antibodies can be induced to cause inflammation and destruction of transplanted tissue.

### Types of Rejection

As discussed above, depending on the organ or tissue transplanted certain particular types of rejection are more common than others. Hyperacute rejection, as suggested by the nomenclature, is a rapid response that occurs within minutes or hours of transplant and can lead to systemic inflammatory responses against the transplanted tissue. Most often, hyperacute rejection is the result of some pre-existing humoral immunity, the transplant serving as a subsequent exposure to nonself antigens. Hyperacute rejection is most often treated by removal of the transplanted tissue.

Acute rejection, with varying degrees of severity, occurs, in essence, in almost all transplants. Acute rejection is tied to the formation of cellular immunity against the transplanted tissue or organ. It typically occurs within 6 to 10 days of transplant, with risk of acute rejection being highest typically in the first 3 to 4 months. However, acute rejection can occur even after longer elapsed times. Acute rejection is most often recognized in highly vascularized tissues that are transplanted, such as, for example, the liver or the kidney. While acute rejection can be recognized and fairly promptly treated, which leads to prevention or reduction in risk of organ failure, recurrent episodes of acute rejection can lead to chronic rejection.

Chronic rejection refers to the long-term loss of function in transplanted organ. In some instances this occurs via fibrosis of the transplanted tissue blood vessels, and subsequent loss of adequate blood and/or oxygen flow to the tissue. Chronic rejection is typified by initial infiltration of lymphocytes which can lead to epithelial cell injury, followed by inflammatory lesions, and potential recruitment of fibroblasts which lead to the formation of scar tissue. Scar tissue, in many organs, obstructs function and/or blood flow which can lead to failure of the transplanted organ and/or further inflammatory or immune responses against the transplanted organ.

Depending on the type of rejection, the methods by which the rejection is diagnosed may vary. For example hyperacute rejection is often noticed and treated in a very short term. Diagnosis of acute and often chronic rejection, relies on clinical data, such as for example, patient symptoms or physical exams. More often than not, however, laboratory data, such as, for example, tissue biopsies and subsequent histochemical or pathological analysis, are used in diagnosing tissue or organ rejection. For example, a tissue biopsy may be evaluated for histological signs of rejection. These may include, but are not limited to, evidence of infiltration of T cells (and/or other cells such as eosinophils or neutrophils). Also indicative of rejection are structural changes to the transplanted tissue anatomy that suggest insufficient blood or nutrient supply, and/or damage due to host immune response. Also, injury to blood vessels, such as that caused by pro-inflammatory reactions, may be indicative of tissue rejection. However, tissue biopsy may be limited, depending on, for example, the health status of a recipient, the potential invasiveness of the biopsy (e.g., depending on what tissue or organ was transplanted).

### Diagnostic Tests

Currently, many diagnostic tests are performed on a biological fluid sample (e.g., blood, urine, etc.) extracted from a patient for the diagnosis or prognosis of disease. The diagnosis or prognosis may be derived from identification of a biomarker or a biochemical pattern that is not present in healthy patients or is altered from a previously obtained patient sample. In several embodiments, the diagnostic tests rely on the presence of known and well characterized biomarkers in the fluid sample (e.g., electrolytes, urea, creatinine, glucose, plasma proteins such as albumins, immunoglobulins and the like, biological compounds such as thiamin, riboflavin, niacin, vitamin B6, folic acid, vitamin D, biotin, or iron). In several embodiments, the diagnostic tests are directed to detection of specific biomarkers (e.g., cell surface proteins) that are unique to diseased cells. In several embodiments, diagnostic tests are designed to detect or identify disease states through the isolation and amplification of nucleic acids, in order to study expression levels of certain disease-associated genes. For example, in several embodiments the methods disclosed herein evaluate the change in expression level of certain markers associated with liver function and/or liver health in order to assess the status of a liver transplant patient (e.g., presence or absence of rejection of the transplanted liver, and severity of the same). In several embodiments, these diagnostic tests employ a file sample isolated or obtained from the recipient of a liver transplant.

Often, use of bodily fluids to isolate or detect biomarkers significantly dilutes a biomarker and results in readouts that lack the requisite sensitivity. Additionally, most biomarkers are produced in low or even moderate amounts in tissues other than the diseased tissue, such as normal tissues. Thus, as described in more detail below, in several embodiments devices are used that enable the concentration of a target nucleic acid (or other biomarker) from a fluid sample such as for example, a bile sample obtained from a liver transplant recipient.

### Vesicle-Associated RNA

As discussed in more detail below, several embodiments of the methods disclosed herein are based on the identification of specific nucleic acids that are markers of disease or injury to the liver. In particular, several embodiments of the methods employ what is generally considered a medical waste material, e.g., bile. Advantageously, in several embodiments, the methods disclosed herein provide a higher degree of sensitivity than alternative diagnostic assays disclosed above. While several embodiments disclosed herein are directed to the isolation of RNA associated with vesicles present in patient bile samples, in several embodiments, RNA (and the associated markers) that are normally found in blood or plasma are isolated from bile samples. In some embodiments, these markers are present in the bile due to damage or disease of the liver, or for example, rejection of a transplanted liver, and are indicative of one or more of the function, rejection status, and general health of the liver.

In several embodiments disclosed herein, there are provided methods for the capture of RNA from a sample of patient body fluid and subsequent analysis of that RNA for disease and/or tissue specific markers. In several embodiments, the method comprises isolation of vesicles associated with RNA from a patient bile sample (though in other embodiments, vesicles used for assessing the status of the liver can be obtained from plasma, serum, cerebrospinal fluid, sputum, saliva, mucus, tears etc.).

As described below, in some embodiments, the nucleic acids are vesicle-associated. In some embodiments, the nucleic acids detected are indicative of liver status post-transplant (or, in some embodiments, another aspect of liver disease and/or function). In several embodiments, the markers are not normally present in the bile of subject (e.g., their presences is indicative of a transplant rejection). In other embodiments, the marker may normally be present, but is expressed at elevated (or reduced) levels. In some embodiments, the detection of the nucleic acids is associated with severity and/or progression of transplant rejection. In some embodiments, bile is collected and nucleic acids are evaluated over time (e.g., to monitor a patient's response to anti-rejection therapy or progression of rejection).

According to various embodiments, various methods to quantify RNA are used, including Northern blot analysis, RNAse protection assay, PCR, RT-PCR, real-time RT-PCR, RNA sequencing, nucleic acid sequence-based amplification, branched-DNA amplification, ELISA, mass spectrometry, CHIP-sequencing, and DNA or RNA microarray analysis.

RNA (and other nucleic acids) are typically within the intracellular environment. However, certain nucleic acids exist extracellularly. For example, in several embodiments, the methods involve collection and analysis of naked extracellular nucleic acids (e.g., naked RNA) from the bile. This is advantageous in several embodiments because, typically, the extracellular environment that comprises substantial quantities of RNAses leads to rapid degradation of the nucleic acids.

In several embodiments, nucleic acids are associated with extracellular vesicles. In several embodiments, diagnosis and characterization of liver transplant rejection is performed by detection and quantification of specific RNA species from RNA-containing vesicles isolated from patient samples (e.g., bile). In one embodiment, such vesicles are trapped on a filter, thereby allowing RNA extraction from the vesicles. In additional embodiments, centrifugation is used to collect the vesicles.

Nucleic acids can be associated with one or more different types of membrane particles (ranging in size from 50-80 nm), exosomes (ranging in size from 50-100 nm), exosome-like vesicles (ranging in size from 20-50 nm), and microvesicles (ranging in size from 100-1000nm). In several embodiments, these vesicles are isolated and/or concentrated, thereby preserving vesicle associated RNA despite the high RNAse extracellular environment. In several embodiments, the sensitivity of methods disclosed here is improved (vis-à-vis isolation of nucleic acids from tissues and/or collection of naked nucleic acids) based on the use of the vesicle-associated RNA.

A variety of methods can be used, according to the embodiments disclosed herein, to efficiently capture and preserve vesicle associated RNA. In several embodiments, centrifugation on a density gradient to fractionate the non-cellular portion of the sample is performed. In some embodiments, density centrifugation is optionally followed by high speed centrifugation to cause vesicle sedimentation or pelleting. As such approaches may be time consuming and may require expensive and specialized equipment in several embodiments, low speed centrifugation can be employed to collect vesicles. Vesicle capture devices and systems are disclosed in more detail below.

In several embodiments, filtration (alone or in combination with centrifugation) is used to capture vesicles of different sizes. In some embodiments, differential capture of vesicles is made based on the surface expression of protein markers. For example, a filter may be designed to be reactive to a specific surface marker (e.g., filter coupled to an antibody) or specific types of vesicles or vesicles of different origin.

In some embodiments, the markers are unique vesicle proteins or peptides. In some embodiments, the severity of liver transplant rejection is associated with certain vesicle modifications which can be exploited to allow isolation of particular vesicles. Modification may include, but is not limited to addition of lipids, carbohydrates, and other molecules such as acylated, formylated, lipoylated, myristolylated, palmitoylated, alkylated, methylated, isoprenylated, prenylated, amidated, glycosylated, hydroxylated, iodinated, adenylated, phosphorylated, sulfated, and selenoylated, ubiquitinated. In some embodiments, the vesicle markers comprise non-proteins such as lipids, carbohydrates, nucleic acids, RNA, DNA, etc.

In several embodiments, the specific capture of vesicles based on their surface markers also enables a “dip stick” format where each different type of vesicle is captured by dipping probes coated with different capture molecules (e.g., antibodies with different specificities) into a patient urine sample.

In several embodiments, vesicle-associated RNA is captured and RNA markers are detected that correspond to certain markers or groups of markers and/or certain stages of liver rejection. For instance, in several embodiments, markers may be detected that are related to an early stage of acute rejection or early stage of acute infection of a liver, including, but not limited to one or more of IL1B, IL6, IL8, or other markers. Additionally, in several embodiments, markers that correspond to acute rejection or acute infection of a transplanted liver are detected, including, but not limited to one or more of TNF-alpha, FasL, IFNG, granzyme B, CD16 DEFA3, PRG2, or other markers. In several embodiments, markers that correspond to sustained rejection or sustained infection of a transplanted liver are detected, including, but not limited to one or more of IL2, IL4, GMCSF, or other markers. In several embodiments, RNA markers that correspond to a recovery phase after liver transplant or recovery phase from acute rejection or acute infection of a transplanted liver are detected, including but not limited to, one or more of IL10, TGF beta, CTLA4, PD-1, FOXP3, or other markers. Depending on the embodiment, markers from more than one group are detected. For example, markers related to acute rejection or infection may still be detected when a subject is in the recovery phase after a liver transplant, for example due to lag time in changes in gene expression.

Sometimes when one or more of IL1B, IL6 or IL8 is detected using the methodology described herein, liver rejection is ruled out. On the other hand, in some instances rejection may not be ruled out when IL1B, IL6 or IL8 is detected. For instance, in some cases, IL1B, IL6 or IL8 may be detected in combination with a marker from another group of markers that also corresponds to liver rejection or liver infection. At least one of the markers from the group of TNF-alpha, FasL, IFNG, granzyme B, CD16 DEFA3, and PRG2 may be detected in combination with at least one marker from the group of IL1B, IL6 or IL8. Or alternatively, at least one of the markers from the group of IL2, IL4 and GMCSF may be detected in combination with at least one of the markers from the group of IL1B, IL6 or IL8. In another example, at least one of the markers from the group of TNF-alpha, FasL, IFNG, granzyme B, CD16 DEFA3, and PRG2 may be detected in combination with at least one of the markers from the group of IL2, IL4 and GMCSF. In some instances, the particular phase of liver rejection (or recovery) is corroborated by other methods, as the transition from one phase to the next is not necessarily an acute change, but could be more gradual (e.g., with overlapping marker expression). In some embodiments, the identification of one or more markers allows an individual to specifically pinpoint whether a liver is subject to rejection or infection, and what phase the rejection or infection is in. In some instances, the identification of one or more markers may allow an individual to rule out rejection or infection.

### Methodology

Free extracellular RNA is quickly degraded by nucleases, making it a potentially poor diagnostic marker. As described above, some extracellular RNA is associated with particles or vesicles that can be found in various biological samples, such as bile excreted from the liver. This vesicle associated RNA, which includes mRNA, is protected from the degradation processes in the bile. Microvesicles are shed from most cell types and consist of fragments of plasma membrane. Microvesicles contain RNA, mRNA, microRNA, and proteins and mirror the composition of the cell from which they are shed. Exosomes are small microvesicles secreted by a wide range of mammalian cells and are secreted under normal and pathological conditions. These vesicles contain certain proteins and RNA including mRNA and microRNA. Several embodiments evaluate nucleic acids such as small interfering RNA (siRNA), tRNA, and small activating RNA (saRNA), among others.

In several embodiments the RNA isolated from vesicles from the bile of a patient with liver transplant rejection is used as a template to make complementary DNA (cDNA), for example through the use of a reverse transcriptase. In several embodiments, cDNA is amplified using the polymerase chain reaction (PCR). In other embodiments, amplification of nucleic acid and RNA may also be achieved by any suitable amplification technique such as nucleic acid based amplification (NASBA) or primer-dependent continuous amplification of nucleic acid, or ligase chain reaction. Other methods may also be used to quantify the nucleic acids, such as for example, including Northern blot analysis, RNAse protection assay, RNA sequencing, RT-PCR, real-time RT-PCR, nucleic acid sequence-based amplification, branched-DNA amplification, ELISA, mass spectrometry, CHIP-sequencing, and DNA or RNA microarray analysis.

In several embodiments, rejection of a transplanted liver by the recipient (or other issues with the received liver, such as infection, relapse of original disease, etc.) induces the expression of one or more markers. In several embodiments, the increased expression is measured by the amount of mRNA encoding said markers (in other embodiments, DNA or protein are used to measure expression levels). In some embodiments bile is collected from a patient and directly evaluated. In some embodiments, vesicles are concentrated, for example by use of filtration or centrifugation. Isolated vesicles are then incubated with lysis buffer to release the RNA from the vesicles, the RNA then serving as a template for cDNA which is quantified with methods such as quantitative PCR (or other appropriate amplification or quantification technique). In several embodiments, the level of specific marker RNA from patient vesicles is compared with a desired control such as, for example, RNA levels from a healthy patient population, or the RNA level from an earlier time point from the same patient or a control gene from the same patient.

In several embodiments, the disclosed methods allow the detection of various clinical problems with a transplanted liver (e.g., rejection, infection, relapse of original disease, etc.) by measuring the levels of mRNA encoding one or more markers related to various liver functions. In several embodiments, the disclosed methods allow the assessment of the progression (or regression) of liver transplant by measuring the levels of mRNA encoding one or more markers related to liver function. To determine these mRNA levels, in some embodiments, mRNA-containing vesicles are isolated from bile using a device for isolating and amplifying mRNA, such as those described above. Additional devices that can also be used for at least part of the isolation and/or amplification process are described in more detail in U.S. Pat. Nos. 7,745,180, 7,939,300, 7,968,288, 7,981,608, 8,076,105, 8,101,344, each of which is incorporated in its entirety by reference herein.

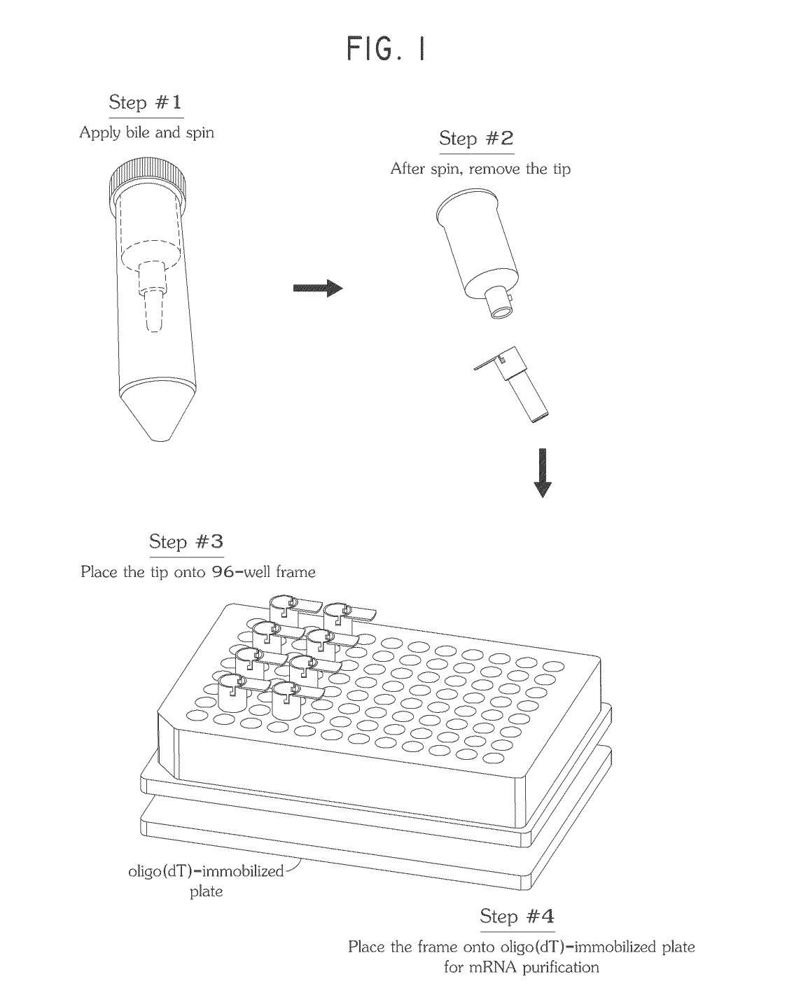

FIG. 1 shows a general schematic of one embodiment of a process for capturing vesicles from a bile sample and preparing the samples for subsequent analysis. In brief, a bile sample is loaded into a vesicle capture device (discussed in more detail below) and centrifuged (though other forces can be applied in other embodiments). Centrifugation causes the bile to pass through a vesicle capture membrane, wherein the vesicles are retained on the membrane and the remainder of the bile (now vesicle-depleted) passes on to the bottom of the centrifuge tube (also referred to as the receiving vessel, depending on the embodiment). Thereafter, the internal portion of the capture device is separated and the filter-containing portion is placed in communication with a multi-well microplate (a 96-well plate is depicted, though other size plates can be used). A lysis buffer is added to each individual vesicle capture portion (e.g., each portion is in an individual well of the microplate) in order to release RNA from the captured exosomes. Thereafter, the RNA is transferred to a plate comprising, for example immobilized oligo-dT, in order to allow subsequent production of complementary DNA (cDNA) and analysis of marker expression for markers related to liver function, liver rejection, liver infection, etc.

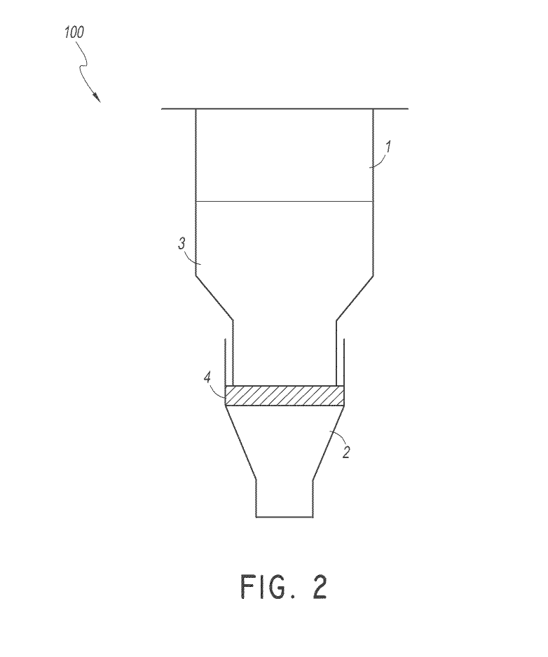

FIG. 2 depicts additional details regarding one embodiment of a capture device 100 used for capturing vesicles from patient bile samples. The embodiment of capture device 100 depicted in FIG. 2 comprises a first hollow body 1 in functional communication with a second hollow body 2. “Functional communication” shall be given its ordinary meaning and shall also refer to the two hollow bodies being coupled in such a manner that bile can pass from the first hollow body to the second hollow body.

For example, in several embodiments, a bile sample 3 is loaded into first hollow body 1 and passed to second hollow body 2, thereby passing through a capture material 4. In some embodiments, capture material 4 retains at least some of the target vesicles contained in the bile sample, for example vesicles comprising nucleic acid or protein that can be used to assess the current physiological state of a subject's liver.

In some embodiments, the capture material (glass fiber filter in some embodiments) is located within second hollow body 2. In several embodiments, after the bile sample has passed through capture material 4, second hollow body 2 is removed from first hollow body 1, and second hollow body 2 is then processed to retrieve the vesicles retained in capture material. In at least one embodiment, exosomes that have been retained by capture material 4 are subsequently recovered from capture material 4 by passing a small amount of liquid (e.g., a lysis buffer) through capture material 4. In some embodiments, another solution (e.g., a washing buffer) is optionally passed through capture material 4 before and/or after application of the liquid used to recover the retained exosomes.

In some embodiments, gravitational force, positive, or negative pressure drives the bile sample through capture material 4. However, in several embodiments, no negative or positive pressure is used, rather, in several embodiments, centrifugal force drives the bile sample through capture material 4. In some embodiments, a wicking-type material drives the bile sample 3 through capture material 4. In some embodiments, capillary action drives the bile sample through capture material 4.

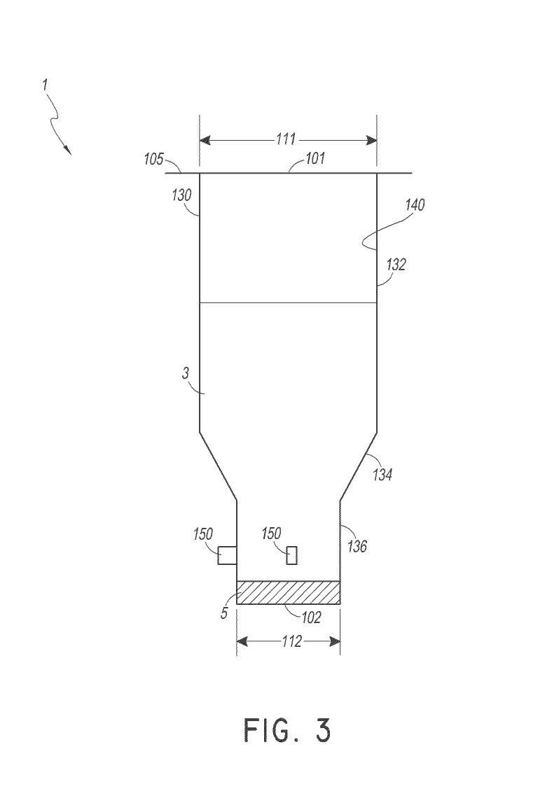

FIG. 3 depicts additional details found in one embodiment of first hollow body 1. In several embodiments, first hollow body 1 has an inlet opening 101, an outlet opening 102, an outer surface 130, and an inner surface 140. In some embodiments, inlet opening 101 is a circular opening having an inlet diameter 111. In some embodiments, outlet opening 102 is a circular opening having an outlet diameter 112. In several embodiments, inlet opening 101 and outlet opening 102 are circular openings that are axially-aligned, with outlet diameter 112 being smaller than inlet diameter 111.

In some embodiments, first hollow body 1 comprises an upper region 132, an intermediate region 134, and a terminal region 136. In some embodiments, upper region 132 and terminal region 136 are cylindrical or substantially cylindrical, and intermediate region 134 is tapered (e.g., conical). In some embodiments, the taper of intermediate region 134 is configured to facilitate passage of a bile sample through outlet opening 102. In some embodiments, first hollow body 1 includes a collar 105 that extends beyond outer surface 130 of an adjacent portion of first hollow body 1. In some embodiments, collar 105 is configured to support first hollow body 1 when first hollow body 1 is inserted into a storage rack or a receiving vessel (not shown).

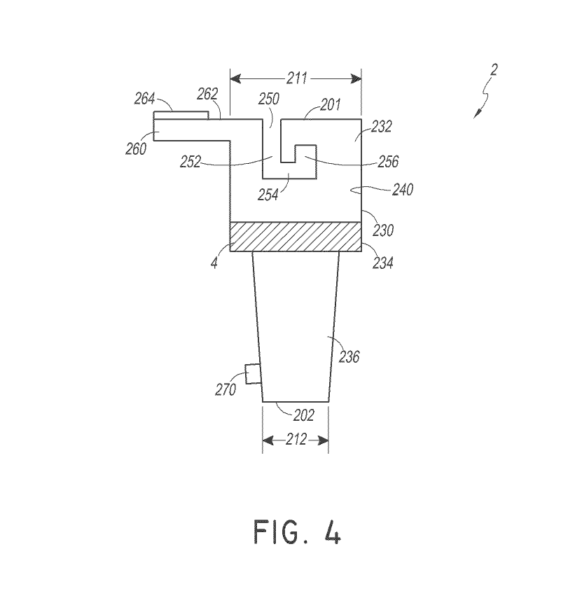

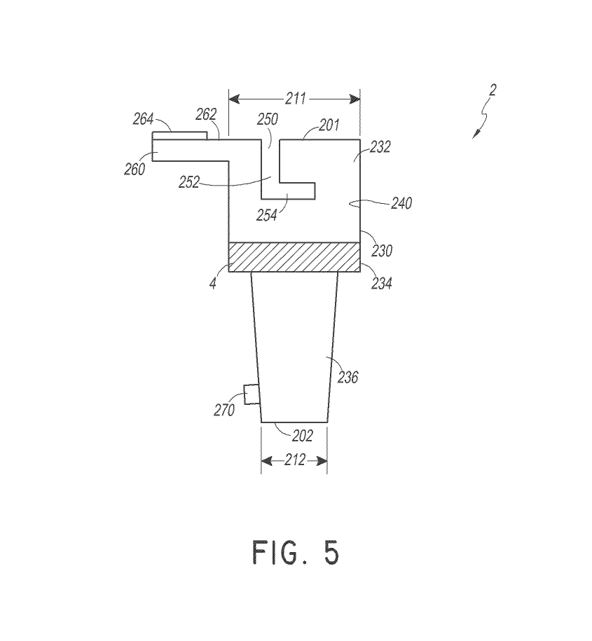

FIG. 4 depicts an embodiment of second hollow body 2. In several embodiments, second hollow body 2 has an inlet opening 201, an outlet opening 202, an outer surface 230, and an inner surface 240. In some embodiments, inlet opening 201 is a circular opening having an inlet diameter 211. In some embodiments, outlet opening 202 is a circular opening having an outlet diameter 212. In several embodiments, inlet opening 201 and outlet opening 202 are circular openings that are axially-aligned, with outlet diameter 212 being smaller than inlet diameter 211.

In several embodiments, first hollow body 1 and second hollow body 2 are made of material that has a low binding affinity for nucleic acids and/or for vesicles (thereby increasing the efficiency of capture of vesicles on the filter material. Suitable materials include, but are not limited to, plastics such as polypropylene, polystyrene, and polyethylene, among others. In some embodiments, first hollow body 1 and second hollow body 2 are made of metal or composite material. In some embodiments, inner surfaces 140, 240 are coated with one or more substances that lowers the binding affinity of the surfaces for nucleic acids (and/or vesicles).

In some embodiments, second hollow body 2 comprises an upper region 232, an intermediate region 234, and a terminal region 236. In some embodiments, terminal region 236 is tapered. In at least one embodiment, the taper of terminal region 236 is configured to facilitate passage of fluid sample 3 out of second hollow body 2.

In several embodiments, second hollow body 2 has a tab 260 that extends from outer surface 230. In some embodiments, tab 260 is located in upper region 232. Tab 260 has an upper surface 262. In some embodiments, upper surface 262 is substantially co-planar with inlet opening 201. In several embodiments, upper surface 262 is sufficiently dimensioned to serve as a platform for labeling second hollow body 2. In at least one embodiment, upper surface 262 is between about 1 mm to about 5 mm wide and about 1 mm to about 5 mm long. In some embodiments, a label 264 is affixed to upper surface 262. In several embodiments, upper surface 262 is marked by any suitable means including ink, or etching. In at least one embodiment, label 264 or the marking of upper surface 262 denotes the identity (e.g., the source patient) of the fluid sample 3 that has been passed through second hollow body 2. In some embodiments, label 264 or marking of upper surface 262 encodes a bar code (e.g., a 2D or 3D bar code). In several embodiments, RFID tags or other identifiers may be used to denote the patient identity from which the sample was obtained.

In several embodiments, upper region 232 of second hollow body 2 is configured to functionally communicate with terminal region 136 of first hollow body 1. First hollow body 1 and second body 2 may functionally communicate by any number of ways including but not limited to mating screw threads, a luer fitting, an interference fit, and a compression fitting (though other types of fittings may be used in additional embodiments). In some embodiments, terminal region 136 of first hollow body 1 is configured to fit inside upper region 232 of second hollow body 2. In some embodiments, upper region 232 of second hollow body 2 is configured to fit inside terminal region 136 of first hollow body 1. In some embodiments, at least a portion of outer surface 130 is surrounded by at least a portion of inner surface 240. In some embodiments, at least a portion of outer surface 230 is surrounded by at least a portion of inner surface 140. In some embodiments, outlet diameter 112 is smaller than inlet diameter 211

In some embodiments, first hollow body 1 has at least one pin 150 that protrudes from outer surface 130 of terminal region 136, and second hollow body 2 has at least one channel 250 in upper region 232 of second hollow body 2 (see e.g., FIG. 4). In at least one embodiment, pin 150 is configured to reversibly cooperate with channel 250. Channel 250 has a longitudinal portion 252, a transverse portion 254, and a retrograde portion 256. In some embodiments, first hollow body 1 is coupled to second hollow body 2 by sliding pin 150 into longitudinal portion 252 of channel 250. First hollow body 1 and second hollow body 2 are positioned to allow pin 150 to reach transverse portion 254 of channel 250. Second hollow body 2 is then rotated to bring pin 150 into transverse portion 254 until pin 150 lines up with retrograde portion 256 of channel 250. The compressive force between first hollow body 1 and second hollow body 2 is then reduced, allowing pin 150 to slide into retrograde portion 256, thereby securing a coupling between first hollow body 1 and second hollow body 2. In some embodiments, second hollow body 2 is removed from first hollow body 1 by squeezing the two hollow bodies together and allowing pin 150 to retrace channel 250.

In some embodiments, the at least one channel 250 in upper region 232 of second hollow body 2 comprises a longitudinal portion 252 and a transverse portion 254. In some embodiments, first hollow body 1 is coupled to second hollow body 2 by sliding pin 150 into longitudinal portion 252 of channel 250. First hollow body 1 and second hollow body 2 are positioned to allow pin 150 to reach transverse portion 254 of channel 250. Second hollow body 2 is then rotated to bring pin 150 into transverse portion 254 thereby securing a coupling between first hollow body 1 and second hollow body 2. After processing, second hollow body 2 is removed from first hollow body 1 by rotating the two hollow bodies in the opposite direction and allowing pin 150 to retrace channel 250, thereby allowing the first and second hollow bodies to disengage.

In several embodiments, capture material 4 is made from any suitable material that can retain the vesicles to be captured from the bile sample. In several embodiments, the material used for capture material 4 is optimized to balance the attractive nature of the material for the vesicles (or naked nucleic acids and/or proteins) and the ability of the material to release the target component under appropriate conditions (e.g., lysis and/or elution).

In some embodiments, capture material 4 is optionally modified to tailor the profile of the vesicles retained by capture material 4. In some embodiments, capture material 4 is electrocharged (e.g., electrostatically charged), coated with hydrophilic or hydrophobic materials, chemically modified, and/or biologically modified. In several embodiments, the zeta potential of capture material 4 is used as a basis for modification (e.g., electrostatic charging) of the material. In some embodiments, capture material 4 (based on its zeta potential) does not require modification. In some embodiments, capture material 4 is modified by attaching a nucleotide sequence to the surface of capture material 4. In some embodiments, a protein is attached to the surface of capture material 4. In some embodiments, biotin or streptavidin is attached to the surface of capture material 4. In some embodiments, an antibody or antibody fragment is attached to capture material 4. Any of such embodiments can be employed to advantageously increase the efficiency of capture of a target.

In some embodiments, differential capture of vesicles is achieved based on the surface expression of protein markers on the vesicles and a complementary agent on capture material 4 which identifies that marker (e.g., an antibody that recognizes an antigen on a particular vesicle). In some embodiments, the markers are unique vesicle proteins or peptides. In such embodiments, capture material 4 may be configured in a manner which allows for recognition of specific vesicle modifications that may occur under certain physiologic conditions (e.g., vesicles may be modified in a manner consistent with liver transplant rejection). Modification of the vesicles may include, but is not limited to the addition of lipids, carbohydrates, and other molecules such as acylated, formylated, lipoylated, myristolylated, palmitoylated, alkylated, methylated, isoprenylated, prenylated, amidated, glycosylated, hydroxylated, iodinated, adenylated, phosphorylated, sulfated, and selenoylated, ubiquitinated. In some embodiments, capture material 4 is configured to recognize vesicle markers comprising non-proteins such as lipids, carbohydrates, nucleic acids, RNA, mRNA, siRNA, microRNA, DNA, etc.

In some embodiments, the interactions between vesicles and capture material 4 are based on electrostatic interaction, hydrophobic interaction, van der Waals force, or a combination of these interactions.

In some embodiments, the materials used for capture material 4 may comprise unwanted materials that inhibit the capture of vesicles. Thus, in several embodiments, capture material 4 is pre-treated to remove such inhibitory materials in advance of using the capture material to capture the vesicles. For example, high concentrations of proteins such as albumin may lower the capture efficiency of vesicle capture. In such cases, albumin can be removed by various techniques, such as, for example, passing materials or solutions through or over capture material 4, the materials or solutions comprising a compound (e.g., Blue Trisacryl M resin) with a greater affinity for the albumin than the albumin has for capture material 4. The techniques used to remove contaminants may also include heating, acid bath, basic bath, ultrasonic cleaning, and the like.

In several embodiments, capture material 4 is made of glass-like material. In some embodiments, capture device 100 optionally includes a filter material 5 (shown in FIG. 3) that is configured to filter fluid sample 3 before fluid sample 3 passes through capture material 4. In some embodiments filter material 5 is placed in second hollow body 2 between capture material 4 and inlet opening 201. In some embodiments, filter material 5 is placed in first hollow body 1 between intermediate region 136 and outlet opening 102. In several embodiments, however, no filter material is used.

In several embodiments, combinations of filter material 5 and capture material 4 are used. In some embodiments, capture material 4 comprises a plurality of layers of material. In several embodiments, capture material 4 comprises at least a first layer and a second layer of glassfiber. In some embodiments, a bile sample is passed through filter material 5 to capture components that are about 1.6 microns or greater in diameter. In some embodiments, a bile sample is passed through capture material 4 so as to capture vesicles having a minimum size from about 0.6 microns to about 0.8 microns in diameter, and having a maximum size of less than about 1.6 microns. In several embodiments, the retention rate of capture material 4 is greater than about 50%, about 75%, about 90%, or about 99% for vesicles having a diameter of from about 0.6 microns to about 1.5 microns in diameter. In at least one embodiment, capture material 4 captures vesicles sized from about 0.7 microns to about 1.6 microns in diameter. In at least one embodiment, capture material 4 captures exosomes or other vesicles ranging in size from about 0.020 microns to about 1.0 microns.

In several embodiments, capture material 4 comprises combinations of glass-like and non-glass-like materials. For example, in one embodiment, a non-glass-like material comprising nitrocellulose is used. In some embodiments, capture material 4 comprises glass-like materials, which have a structure that is disordered, or “amorphous” at the atomic scale, such as plastic or glass. Glass-like materials include, but are not limited to, glass beads or fibers, silica beads (or other configurations), nitrocellulose, nylon, polyvinylidene fluoride (PVDF) or other similar polymers, metal or nano-metal fibers, polystyrene, ethylene vinyl acetate or other co-polymers, natural fibers (e.g., silk), alginate fiber, or combinations thereof. Other suitable materials for capture material 4 include zeolite, metal oxides or mixed metal oxides, aluminum oxide, hafnium oxide, zirconium oxide, or combinations thereof.

In some embodiments, vesicles are retained in capture material 4 by virtue of the vesicle having physical dimensions that prohibit the vesicle from passing through the spaces of capture material 4 (e.g., physical retention based on size). In some embodiments, vesicles are retained in capture material 4 by bonding forces between the vesicle and capture material 4. In some embodiments, vesicles form antigen-antibody bonds with capture material 4. In several embodiments, vesicles form hydrogen bonds with capture material 4. In some embodiments, van der Waals forces form between the vesicle and capture material 4. In some embodiments, nucleotide sequences of the vesicle bind to nucleotide sequences attached to capture material 4.

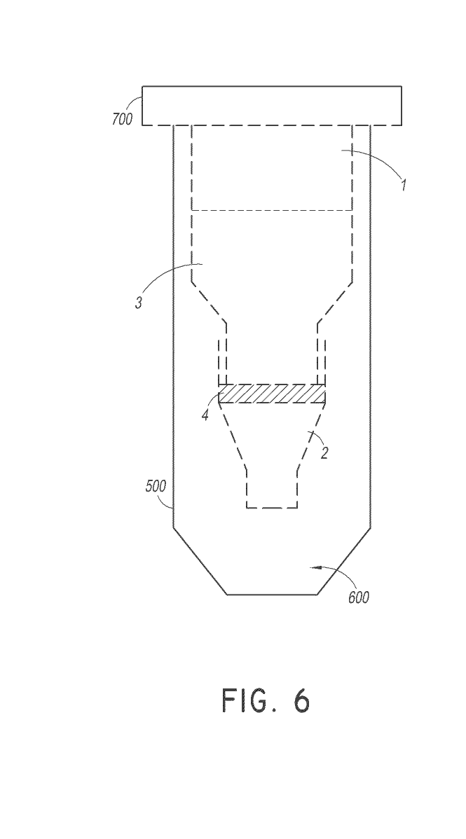

In several embodiments, capture device 100 is used in conjunction with a receiving vessel 500 (see FIG. 6) that receives a bile sample in a receiving compartment 600 after fluid sample 3 has passed through capture device 100. In some embodiments, the receiving vessel also includes a cap 700, to secure the capture device 100 within the receiving vessel 500 during processing. In several embodiments, the cap is a press-fit cap, while in other embodiments the cap comprises a screw-fit cap. In several embodiments, the receiving vessel comprises a centrifuge tube, thus, in some embodiments, first hollow body 1 and second hollow body 2 are sized to fit within a receiving vessel/centrifuge tube. In some embodiments, collar 105 serves as a means for holding capture device 100 in a fixed position relative to the receiving vessel. In several embodiments, capture device 100 and collar 105 are sized to permit use of capture device 100 with a receiving vessel such as a 10 mL, 12 mL, 15 mL, 30 mL, 50 mL, 175 mL, or 225 mL centrifuge tube, though centrifuge tubes of other sizes and capacities are also contemplated. In some such embodiments, collar 105 is sized to fit over the mouth of the centrifuge tube without obstructing the function of the threaded cap of the centrifuge tube. In several embodiments, capture device 100 is placed within a centrifuge tube, and centrifugal force is applied to drive fluid sample 3 from first hollow body 1 through capture material 4 and into second hollow body 2.

In some embodiments, capture device 100 is sized so that outlet opening 202 of second hollow body 2 does not contact fluid sample 3 after fluid sample 3 has passed through capture device 100 and accumulated in the receiving vessel. In some embodiments, the volume capacity of the receiving vessel is greater than the volume capacity of capture device 100 by about 2-fold, by about 3-fold, by about 4-fold, or by about 5-fold.

In some embodiments, capture device 100 has a volume sufficient to receive a bile sample and optionally other reagents to facilitate binding of vesicles and/or nucleic acids to capture material 4. In some embodiments, capture device 100 is sized to accommodate a bile sample volume of between about 1 mL and 1000 mL, including between about 1 mL and 100 mL, between about 5 mL and 50 mL, between about 10 mL and 20 mL, and any volumes between those ranges. In some embodiments, capture device 100 accommodates a volume of about 15 mL.

In some embodiments, the capacity of first hollow body 1 is greater than the capacity of second body 2 by about 100-fold, or by about 50-fold, or by about 20-fold, or by about 10-fold, or by about 5-fold. In some embodiments, the capacity of first hollow body 1 is about the same as the capacity of second hollow body 2.

In many embodiments, the dimensions of capture material 4 are optimized to balance having sufficient capture material 4 to adequately capture vesicles from the bile sample while also allowing a small volume of liquid (e.g., microliter scale) to be used to lyse/elute bound vesicles components. Reducing the volume of recovery liquid allows, in certain advantageous embodiments, the content of vesicles to be extracted at higher concentrations. In some embodiments, the volume of capture device 100 is greater than the volume of capture material 4 by about 1000-fold, by about 500-fold, by about 300-fold, or by about 100-fold. In embodiments where the material of capture material 4 includes interstitial spaces, the meaning of the phrase “volume of capture material 4” shall be taken to include the volume of these interstitial spaces. In several embodiments, the lysis or elution volume ranges from about 5 to about 500 microliters, including about 5 microliters to about 10 microliters, about 10 microliters to about 20 microliters, about 20 microliters to about 50 microliters, about 50 microliters to about 100 microliters, about 100 microliters to about 150 microliters, about 150 microliters to about 200 microliters, about 200 microliters to about 300 microliters, about 300 microliters to about 400 microliters, about 400 microliters to about 500 microliters, and overlapping ranges therebetween.

In some embodiments, capture material 4 is cuboidal. In some embodiments capture material 4 is wafer-shaped, spherical, or some combination thereof. In some embodiments capture material 4 has a surface area to thickness ratio of about 50:1, about 25:1, about 10:1, about 5:1, or about 3:1. In some embodiments, capture material 4 is a cylindrical wafer having a diameter to length ration of about 20:1, about 10:1, about 5:1, or about 2:1. In at least one embodiment, capture material 4 is cylindrical and has a diameter of about 9 mm and a thickness of about 1 mm.

In some embodiments, terminal region 236 of second hollow body 2 is sized to fit within a well of a standard multi-well plate. In several embodiments, terminal region 236 is sized to fit within a well of a standard 6-well plate, or a standard 12-well plate, or a standard 24-well plate, or a standard 96-well plate, or a standard 384-well plate, or a standard 1536-well plate, etc. Such plates are commercially available from various manufacturers, including but not limited to, Corning, Nunc, Fisher, BD Biosciences, etc. In several embodiments, the plates have well dimensions that are shown in Table 1.

TABLE 1
Example Microplate Dimensions for Use with Capture Systems

| Number | Plate | Plate | Well Diameter |
|---|---|---|---|
| of Wells | Length (mm) | Width (mm) | (mm, at top of well) |
| 6 | 127.76 | 85.47 | 35.43 |
| 12 | 127.89 | 85.6 | 22.73 |
| 24 | 127.89 | 85.6 | 16.26 |
| 48 | 127.89 | 85.6 | 11.56 |
| 96 | 127.8 | 85.5 | 6.86 |

In several embodiments, a “carrier” or frame is used to facilitate the stable positioning of the second hollow body in a well of a microplate. In several embodiments, the frame has the same number of wells as the microplate, and functions to align a particular second hollow body with a corresponding well in the microplate. In several embodiments, the frame is removed after the lysis and transfer of the nucleic acid content of the vesicles to the microplate. In several embodiments, the microplate is treated so that is has immobilized oligo(dT) in each well of the microplate.

In some embodiments, tab 260 of second hollow body 2 extends over at least a portion of a neighboring well of a multi-well plate when second hollow body 2 interacts with a first well of the multi-well plate. In at least one embodiment, tab 260 is configured to allow half of the wells of a multi-well plate to be occupied at a time by second hollow bodies 2 without tabs 260 overlapping with one another. In some embodiments, second hollow body 2 has a protrusion 270 that interacts with a wall of a well of a multi-well plate and secures second hollow body 2 to a well of the multi-well plate. In several embodiments, tab 260 is dimensioned so that each well of a multi-well plate can be used to receive a sample.

In several embodiments, a method for isolating a biomarker comprises taking a fluid sample 3 from a patient, passing the fluid sample 3 through capture material 4, removing non-vesicle material from capture material 4, and lysing the vesicles in or on capture material 4 with a lysis buffer, thereby isolating a biomarker from the vesicles. In some embodiments, the biomarker is selected from the group consisting of RNA, DNA, protein, and carbohydrate. In several embodiments, the RNA is of a type selected from the group consisting of mRNA, miRNA, rRNA, tRNA, and vRNA.

In some embodiments, capture device 100 is placed within a centrifuge tube, and collar 105 holds capture device 100 in a fixed position relative to the centrifuge tube. Fluid sample 3 is loaded into capture device 100 before or after placing capture device 100 within the centrifuge tube. Capture device 100 is subjected to centrifugation. The centrifuge tube serves as a receiving vessel and receives fluid sample 3 after it has passed through capture device 100. In some embodiments, low-speed centrifugation is used to drive fluid sample 3 through capture device 100.

In several embodiments, each second hollow body is positioned in a well of a microplate (either with or without use of a carrier/frame) and the captured vesicles on the filter within the second hollow body are then lysed with a lysis buffer, thereby releasing RNA from the captured vesicles. The RNA is then transferred to the microplate (e.g., by centrifugation and/or vacuum pressure). Optionally, the wells of the microplate are treated with oligo(dT) that is immobilized in the well, such that the RNA will hybridize to the well of the microplate via the oligo(dT). In such embodiments, the RNA-oligo(dT) complex can be washed, with the RNA being retained within the well of the plate. Further detail regarding the composition of lysis buffers that may be used in several embodiments can be found in U.S. Pat. No. 8,101,344, which is incorporated in its entirety by reference herein. In several embodiments, cDNA is synthesized from the oligo(dT)-immobilized RNA. In some embodiments, the cDNA is then amplified using real time PCR with primers specifically designed for amplification of liver function or disease-associated markers. Primers that are used in such embodiments are shown in Table 2. Further details about the PCR reactions used in some embodiments are also found in U.S. Pat. No. 8,101,344, which is incorporated in its entirety by reference herein.

TABLE 2
Primer Sequences for RT-PCR Amplification

| | SEQ ID | | SEQ ID | |
|---|---|---|---|---|
| Target | No. | FWD Sequence (5′-3′) | No. | REV Sequence (3′-5′) |
| β- | 1 | CCTGGCACCCAGCACAAT | 2 | GCCGATCCACACGGAGT |
| Actin | | | | ACT |
| ALB | 3 | TGCAAGGCTGACGATAAGGA | 4 | GTAGGCTGAGATGCTTT |
| | | | | TAAATGTGA |
| HGF | 5 | TCCACGGAAGAGGAGATGAGA | 6 | TCATTAAAACCAGATCT |
| | | | | GATCCTTCA |
| VEGF | 7 | CGCAGCTACTGCCATCCAAT | 8 | TGGCTTGAAGATGTACT |
| | | | | CGATCTC |
| IL1B | 9 | GAAGATGGAAAAGCGATTT | 10 | GGGCATGTTTTCTGCTTG |
| | | GTCTT | | AGA |
| IL2 | 11 | GAACTAAAGGGATCTGAAA | 12 | TGTTGAGATGATGCTTT |
| | | CAACATTC | | GACAAAA |
| IL4 | 13 | CACAGGCACAAGCAGCTGAT | 14 | CCTTCACAGGACAGGAA |
| | | | | TTCAAG |
| IL6 | 15 | TCATCACTGGTCTTTTGGAG | 16 | TCTGCACAGCTCTGGCT |
| | | TTTG | | TGT |
| IL8 | 17 | TGCTAAAGAACTTAGATGTC | 18 | TGGTCCACTCTCAATCA |
| | | AGTGCAT | | CTCTCA |
| IL10 | 19 | GCCATGAGTGAGTTTGACAT | 20 | GATTTTGGAGACCTCTA |
| | | CTTC | | ATTTATGTCCTA |
| TNFSF2 | 21 | CGAAGGCTCCAAAGAAGAC | 22 | CAGGGCAATGATCCCAA |
| | | AGT | | AGT |
| TNFSF6 | 23 | TGGCAGCATCTTCACTTCTA | 24 | GAAATGAGTCCCCAAAA |
| | | AATG | | CATCTCT |
| DEFA3 | 25 | CCAGGCTCAAGGAAAAACATG | 26 | CTGGTAGATGCAGGTTC |
| | | | | CATAGC |
| CD16 | 27 | GTTTGGCAGTGTCAACCATCTC | 28 | AAAAGGAGTACCATCAC |
| | | | | CAAGCA |
| GMCSF | 29 | GGCCCCTTGACCATGATG | 30 | TCTGGGTTGCACAGGAA |
| | | | | GTTT |
| IFNG | 31 | GGAGACCATCAAGGAAGAC | 32 | GCTTTGCGTTGGACATT |
| | | ATGA | | CAA |
| PRG2 | 33 | ACTGCGTGGCCCTGTGTAC | 34 | CAGTAGGAACAGATGAA |
| | | | | AGGAAGTCTT |

After the completion of the PCR reaction, the mRNA (as represented by the amount of PCR-amplified cDNA detected) for one or more markers is quantified. In certain embodiments, quantification is calculated by comparing the amount of mRNA encoding a liver marker to a reference value. In some embodiments the reference value will be the amount of mRNA found in healthy non-diseased patients. In other embodiments, the reference value is the expression level of a house-keeping gene. In certain such embodiments, beta-actin, or other appropriate housekeeping gene is used as the reference value. Numerous other house-keeping genes that are well known in the art may also be used as a reference value. In other embodiments, a house keeping gene is used as a correction factor, such that the ultimate comparison is the expression level of marker from a diseased patient as compared to the same marker from a non-diseased (control) sample. In several embodiments, the house keeping gene is a tissue specific gene or marker, such as those discussed above. In still other embodiments, the reference value is zero, such that the quantification of the markers is represented by an absolute number. In several embodiments a ratio comparing the expression of one or more markers from a diseased patient to one or more other markers from a non-diseased person is made.

In some embodiments, a kit is provided for extracting target components from fluid sample 3. Kits often allow better management of quality control and better consistency in results. In some embodiments, a kit comprises a capture device 100 and additional items useful to carry out methods disclosed herein. In some embodiments, a kit comprises reagents selected from the group consisting of lysis buffers, chaotropic reagents, washing buffers, alcohol, detergent, or combinations thereof. In some embodiments, kit reagents are provided individually or in storage containers. In several embodiments, kit reagents are provided ready-to-use. In some embodiments, kit reagents are provided in the form of stock solutions that are diluted before use. In some embodiments, a kit comprises plastic parts that are useful to carry out methods herein disclosed. In some embodiments, a kit comprises plastic parts selected from the group consisting of racks, centrifuge tubes, vacuum manifolds, and multi-well plates. Instructions for use are also provided, in several embodiments.

In several embodiments, the analyses described herein are applicable to human patients, while in some embodiments, the methods are applicable to animals (e.g., veterinary diagnoses).

### Rejection Therapies

When the methods disclosed herein are employed, in several embodiments, they enable a medical professional to make a more patient-specific and diagnosis and symptom-tailored treatment plan, if needed. For example, in several embodiments wherein liver rejection is detected, various rejection therapies can be investigated and/or implemented. For example, if early stage chronic rejection is detected by way of increased or decreased expression of rejection-associated markers, a retransplant can be considered. Acute rejection may be treated with mmunosuppressive therapy (e.g., corticosteroids, calcineurin inhibitors, anti-proliferative agents, mTOR inhibitors, ciclosporin, tacrolimus, azathioprine, mycophenolic acid, sirolimus, everolimus, and combinations thereof can be administered. In several embodiments, antibody-based treatments can be employed to supplement (or replace) immunosuppressive therapy. Antibody drugs may include, monoclonal anti-IL-2Rα receptor antibodies, basiliximab, daclizumab, anti-thymocyte globulin (ATG), anti-lymphocyte globulin (ALG), monoclonal anti-CD20 antibodies, rituximab. In severe cases, blood transfusion may be given to those subjects who are refractory to immunosuppressive or antibody therapies. Also, in several embodiments, bone marrow transplant may be employed, for example, replacement of the transplant recipient's immune system with the donor's, thereby allowing the recipient to accept the liver without rejection. The systems, methods, and devices disclosed herein facilitate the diagnosis and treatment of such clinical situations.

In some instances, diagnosis of a patient or subject is based on the result of RNA markers identified from vesicle-associated RNA collection. In some instances, the RNA markers detected indicate that a patient is in an early phase, acute phase, or sustained phase of liver rejection or liver infection. Based on the phase of rejection, a medical professional or other individual may administer an appropriate treatment. In some instances when the patient or subject is determined to be in an early phase of rejection, the therapy administered is antibiotic therapy.

In some embodiments, a medical professional may be in need of genetic testing in order to diagnose, monitor and/or treat a patient. Thus, in several embodiments, a medical professional may order a test and use the results in making a diagnosis or treatment plan for a patient. For example, in some embodiments a medical professional may collect a sample from a patient or have the patient otherwise provide a sample for testing. The medical professional may then send the sample to a laboratory or other third party capable of processing and testing the sample. Alternatively, the medical professional may perform processing and testing of the sample himself/herself (e.g., in house). Testing may provide quantitative and/or qualitative information about the sample, including data related to the presence of disease or liver rejection. Once this information is collected, in some embodiments the information may be compared to control information (e.g., to a baseline or normal population) to determine whether the test results demonstrate a difference between the patient's sample and the control. After the information is compared and analyzed, it is returned to the medical professional for additional analysis. Based on the results of the tests and the medical professional's analysis, the medical professional may decide how to treat or diagnose the patient.

### EXAMPLES

### Example 1

### Assessment of Post-Transplant Liver Condition

As discussed above, transplanted organs are subject to numerous potential clinical problems, including but not limited to rejection, infection, relapse of original disease, drug toxicity, etc. Early diagnosis and differential diagnosis are important for the timing of treatment as well as the choice of appropriate drugs and/or drug combinations. Clinical symptoms are often non-specific in nature and do not allow of accurate diagnosis. Biopsy provides a definite diagnosis, but is invasive and generally cannot be routinely performed. The present example demonstrates how the methods disclosed here allow for improved assessment of liver condition post-transplant.

### Methods

In some cases, after a liver transplant, bile is collected for several days (or up to a few weeks) after surgery. In most cases drained bile is considered a medical waste, however the methods disclosed herein advantageously employ this “waste” as a source of information related to the status of a subject's liver.

Six recipients of liver transplantation were studied. Bile was collected from an external drainage tube after liver transplantation. Daily, approximately 5 mL bile was collected in a sterile tube and stored at −80° C. The characteristics of the subjects are summarized in Table 3.

TABLE 3
Liver Transplant Recipient Characteristics

| # | Disease | Age | Gender | Blood type | Donor | Biliary reconstruction procedure |
|---|---|---|---|---|---|---|
| 360 | Biliary atresia | 10 | F | A?A | Live | choledochojejunostomy |
| 361 | Primary biliary cirrhosis | 48 | F | AB?^{A} | Live | choledochocholedochostomy |
| 362 | Hepatitis C, liver cirrhosis, | 50 | M | B?A | Live | choledochocholedochostomy |
| | hapatocellular caricinoma | | | | | |
| 363 | Biliary atresia | 0 | F | O?O | Live | choledochojejunostomy |
| 364 | Biliary atresia | 0 | M | AB?A | Live | choledochojejunostomy |
| 365 | Fluminant hepatitis | 19 | F | A?A | Brain death | choledochocholedochostomy |

Bile (1.5 mL) was mixed with 4 mL 5× PBS to equalize pH and salt concentrations (though in some embodiments, no equalization is required), and mixed vigorously to homogenize mucous materials. The diluted bile solution was applied to an exosome collection device (discussed in detail above) and centrifuged for 5 min at 2000×G at 4° C. Briefly, the dilute bile solution was added to the inlet of a first hollow body that was coupled to a second hollow body that contained an exosome capture membrane. That assembly (first and second hollow bodies) was placed in a centrifuge tube and centrifuged to cause the dilute bile solution to pass through the exosome capture membrane within the second hollow body. The exosome-depleted bile was collected in the bottom of the centrifuge tube, and later discarded (though in some embodiments, the bile could be reloaded back into the first hollow body and passed through the exosome filter one or more additional times, in order to capture additional exosomes). The second hollow body was de-coupled from the first hollow body and placed in a multiwell frame (e.g., a 96-well frame) (see, e.g., FIG. 1).

100 μL of lysis buffer was added to each capture membrane and incubated at 37° C. for 10 minutes to release mRNA from exosomes captured on the membrane. The 96-well frame was then placed onto oligo(dT)-immobilized plate (FIG. 1), and centrifuged for 5 min at 2000×G at 4° C., thereby transferring the mRNA liberated from the exosomes to the corresponding well of the 96-well plate. The resultant mRNA-containing oligo(dT)-immobilized plate was stored at 4° C. overnight to allow hybridization between poly(A)⁺ tail of mRNA and the immobilized oligo(dT) in each well of the plate. Subsequently, the plate was washed with wash buffer several times to remove non-mRNA materials, and cDNA was synthesized on the plate by adding dNTP and reverse transcriptase. The cDNA was used then used for real time PCR to evaluate gene expression of markers associated with liver function. Primer sequences are shown in Table 2 above.

### Results

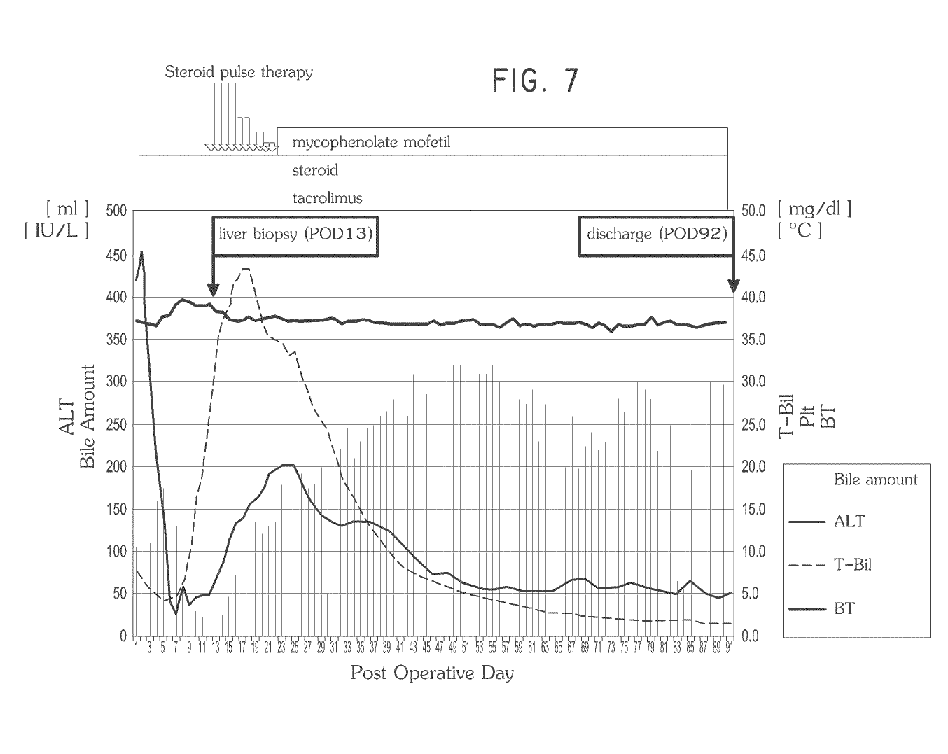

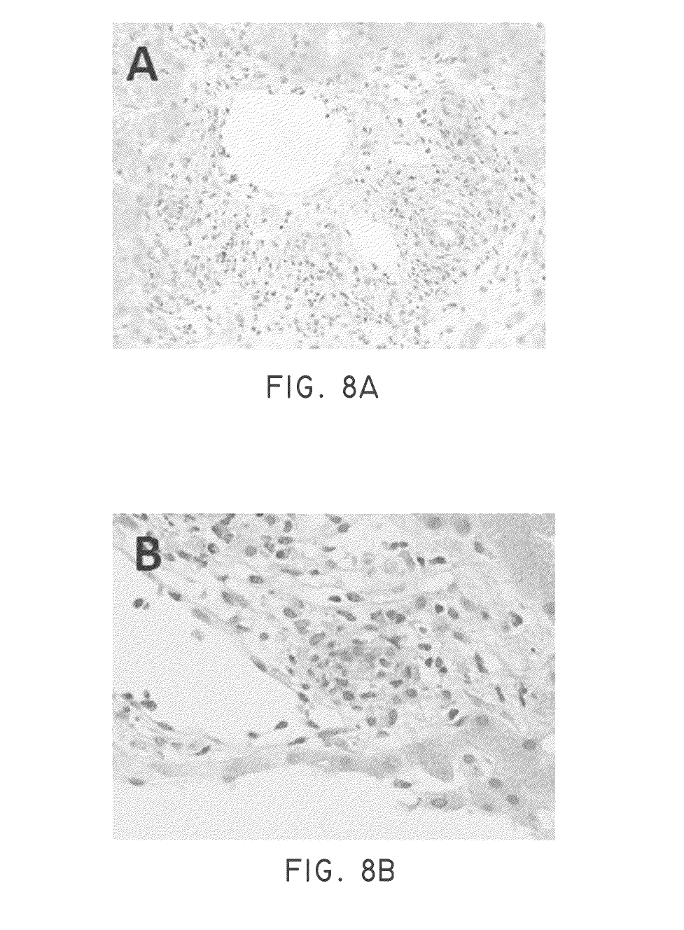

Among 6 patients, a single patient developed acute rejection as shown in FIG. 7. FIG. 7 depicts the body temperature, serum levels of total bilirubin and alanine aminotransferase (ALT), each of which were elevated around 1 week after surgery. The rejection was confirmed by the pathological analysis of biopsy specimens (see FIG. 8) that were scored on the Banff classification rejection activity index (RAI) at 6-8, indicating a moderate to severe rejection. FIG. 8A shows lymphocyte, eosinophils, and neutrophil infiltration to portal area and associated endothelitis. FIG. 8B shows lymphocyte infiltration to bile ducts. After steroid pulse therapy, the physiological parameters were controlled and the subject was discharged on post-operative day 92 (see FIG. 7).

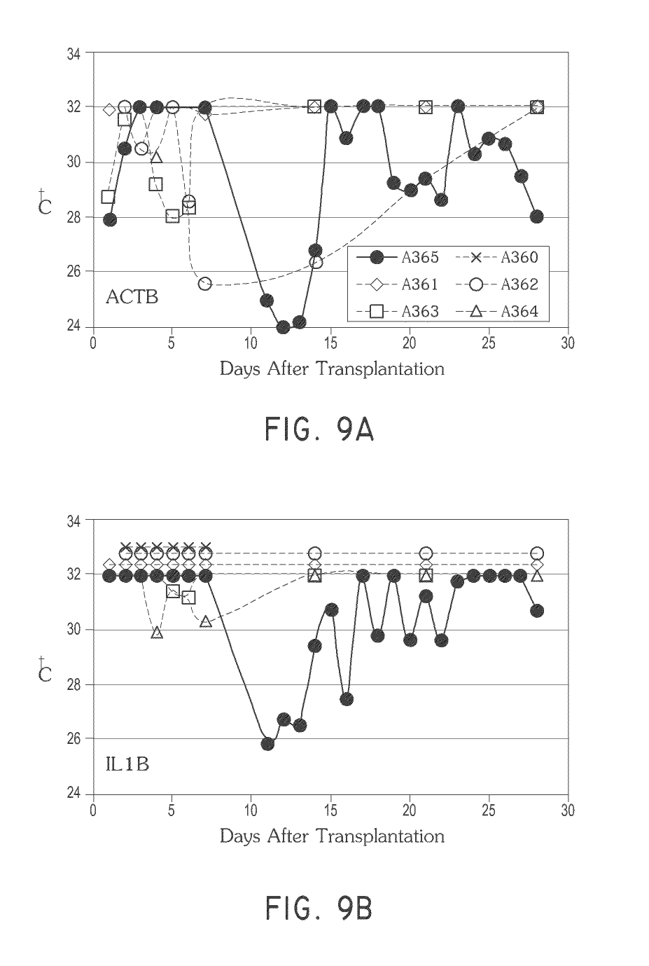

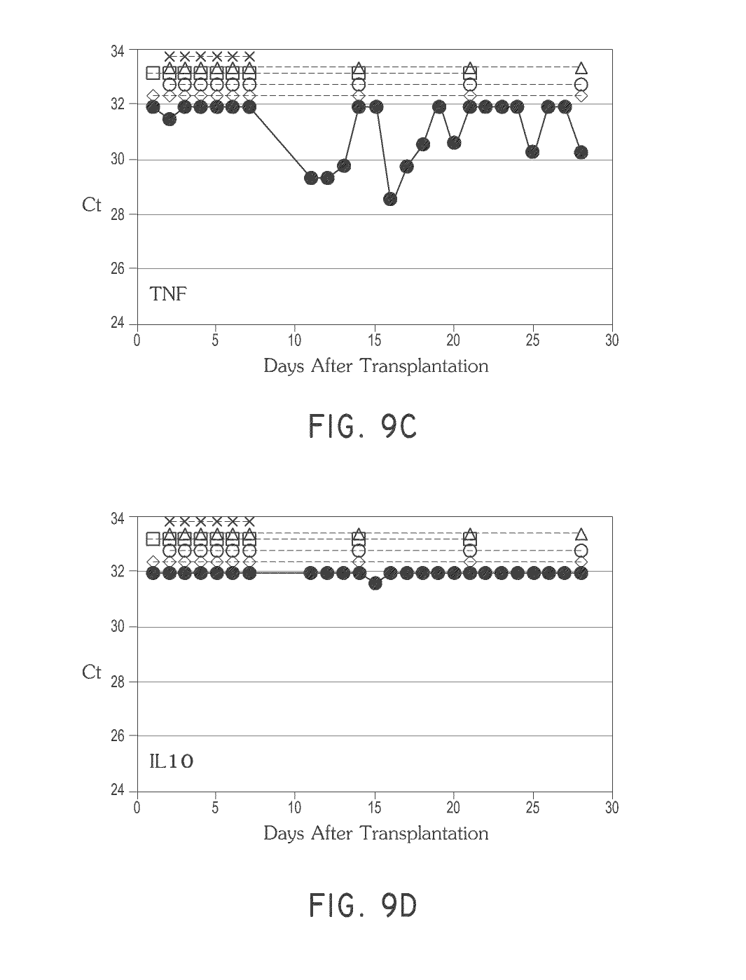

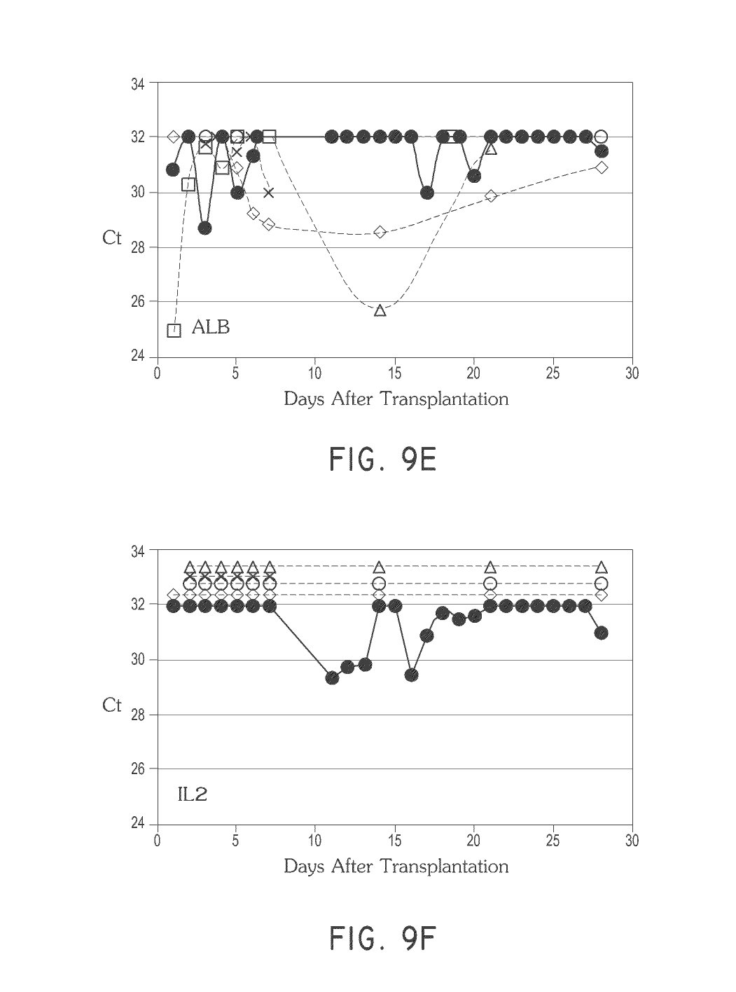

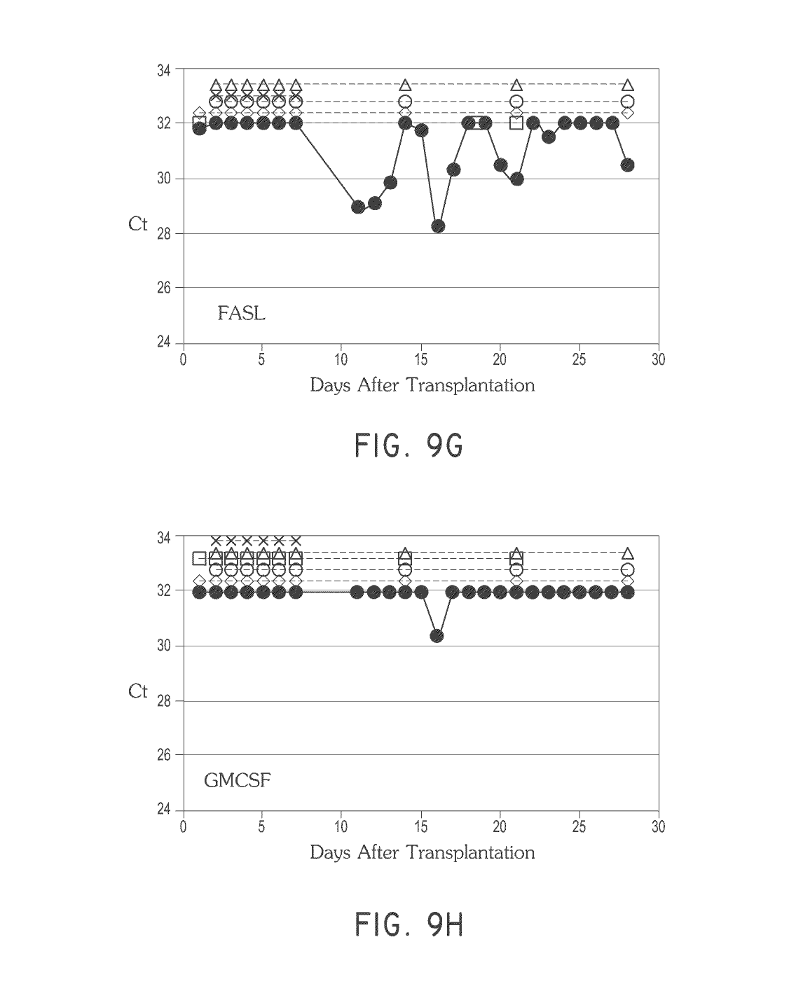

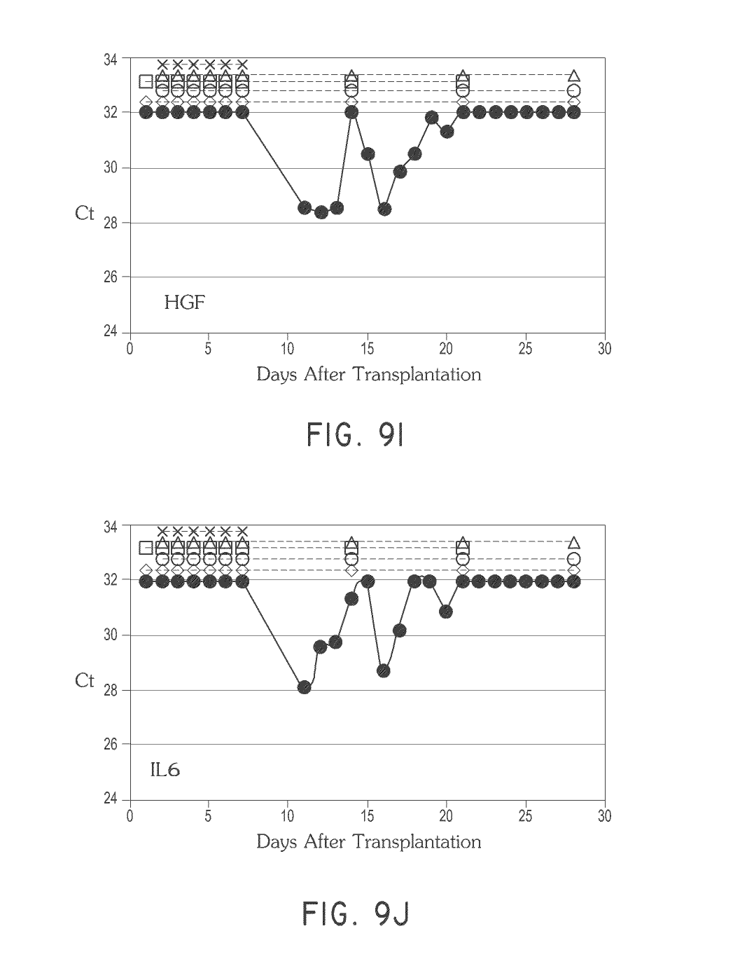

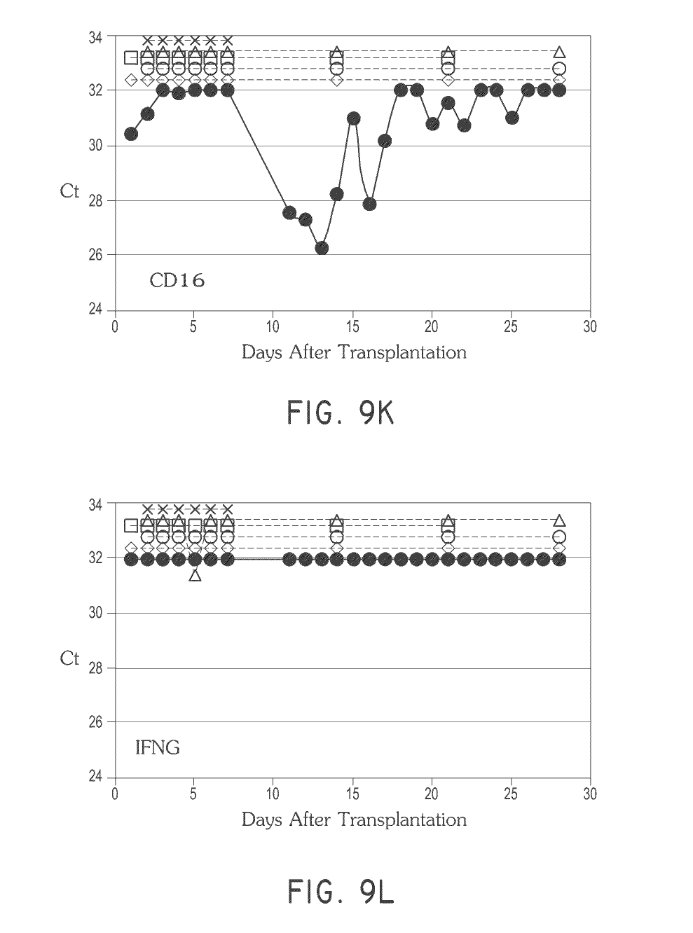

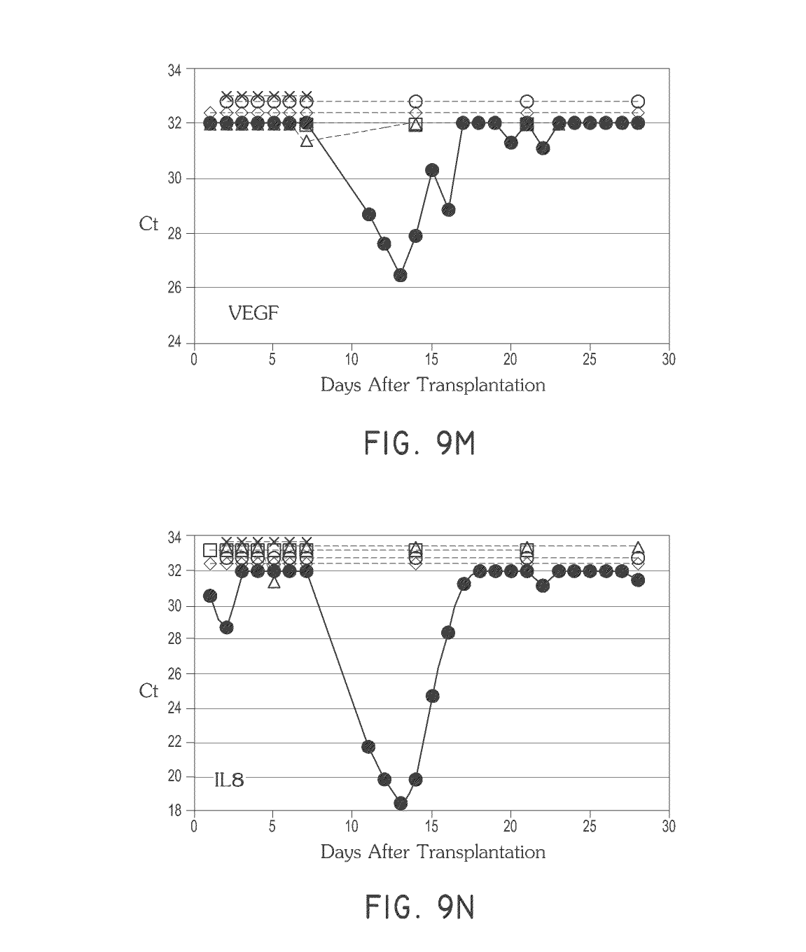

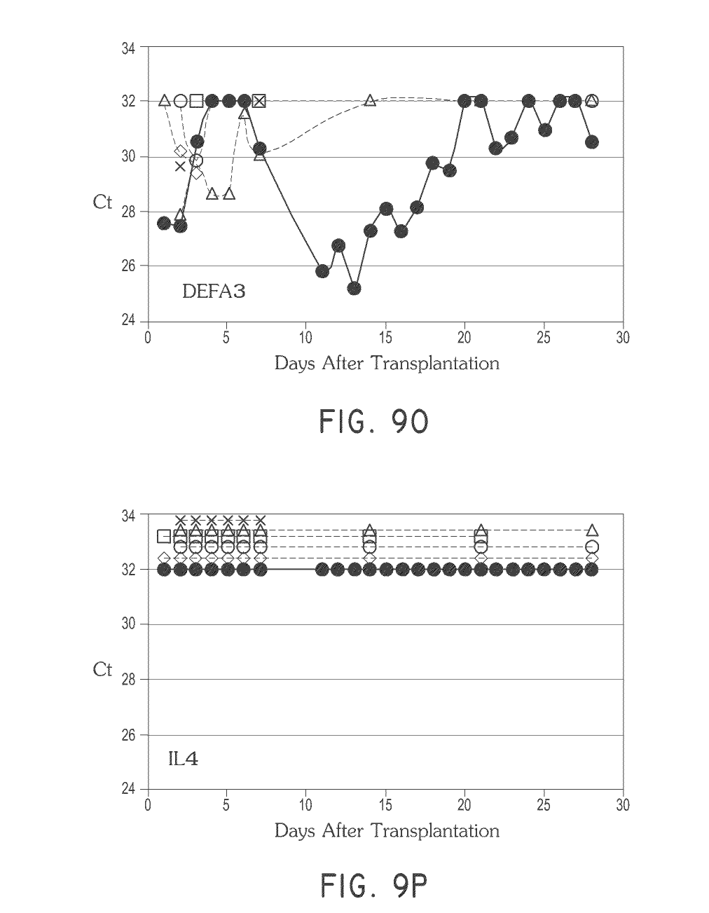

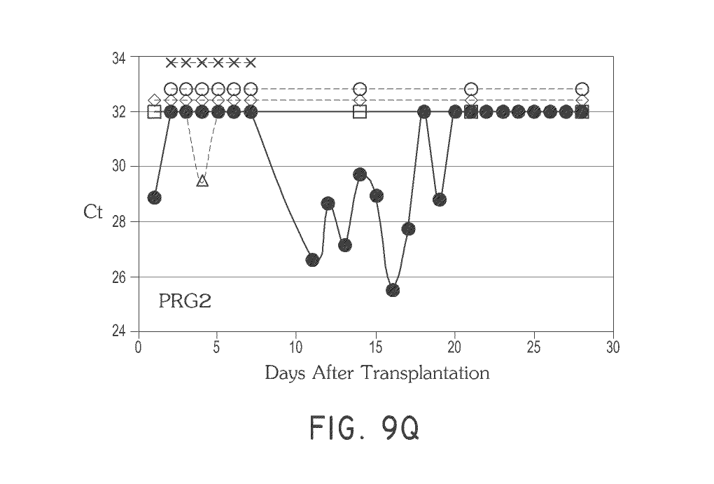

Using the methods for capture and analysis of exosomal mRNA disclosed herein, bile samples from each of the subjects were assessed. As shown in FIG. 9A, the control housekeeping gene (β-actin, ACTB) was detected in bile samples from all patients, thus confirming that bile contained exosomes, and mRNA was preserved in exosomes, despite the harsh condition in bile. ACTB expression levels were not correlated with the development of rejection. Similarly, as shown in FIG. 9E, liver-specific albumin (ALB) mRNA was also detected in the bile exosome. This data confirms that the bile samples contained liver-derived exosomes. However, ALB expression did not correlate to the presence or absence of acute rejection.

In contrast to ACTB and ALB, various chemokine mRNAs were increased at the time of rejection (see FIG. 9B for IL1B, FIG. 9J for IL6, and FIG. 9N for IL8), whereas these mRNAs were undetected in the other patients that did not have acute rejection. Interestingly, the levels of IL8 were very prominent, and higher than ACTB. These data suggest that IL8 may be useful as an early marker of rejection. The induction of IL2 mRNA (FIG. 9F) indicated that immune cascades were activated in the rejected liver. The detection of tumor necrosis factor superfamily (TNFSF) mRNAs (TNFSF2=TNFα, FIG. 9C and TNFRSF6=FasL, FIG. 9G) suggested that cytotoxic T-cell activity was involved in the acute rejection. The detection of hepatic growth factor (HGF, FIG. 9I) and vascular epidermal growth factor (VEGF, FIG. 9M) mRNAs, relate to regrowth of liver tissue and associated vasculature and thus indicate that the recovery process was started in the rejected liver. Since IL4 mRNA was not induced (FIG. 9P), this acute rejection episode was not strong enough to induce immunoglobulin synthesis, which further suggested that the rejection would be controlled by immunosuppressant therapy (see FIG. 7, mycophenolate mofetil administration). Expression levels of the anti-inflammatory cytokine, IL10, were not induced (FIG. 9D). This suggests that the inhibition cascades of immune activation were relatively weak in this patient having acute rejection. Interestingly, neutrophil marker DEFA3 (defensin α3, FIG. 9O) was present in the first 1 week after transplantation in 3 cases, which suggests that there is some degree of neutrophil infiltration after transplantation, even if rejection does not develop. DEFA3 and the eosinophil marker PRG2 (Proteoglycan 2, a natural killer cell activator, eosinophil granule major basic protein, FIG. 9Q) were both induced in the subject having acute rejection. These data correspond to the neutrophil and eosinophil infiltration identified in the biopsy findings (FIG. 8A/8B). CD16 is the marker of NK cells, and the induction of this gene (FIG. 9K) also indicated the contribution of NK cells at the time of rejection. Together, these data indicate that the methods disclosed herein and employed in the present example allow for the capture of exosomes from bile samples, and subsequent isolation and detection of mRNA from the exosomes. Moreover, detection of various immune markers is possible, and are indicative of various aspects of immune activity (or lack thereof) in transplanted livers. As such, the methods disclosed herein allow the assessment of the clinical status of a subject's liver, and in some embodiments, early detection of rejection and/or other disease (e.g., prior to manifestation of clinical symptoms). These methods therefore enable earlier diagnosis of liver maladies and treatment (or prevention) regimes to be implemented in a fashion that results in better clinical outcomes and improved patient care.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a treatment to a subject after determining the subject is suffering from liver transplant rejection” include “instructing the administration of a treatment to a subject after determining the subject is suffering from liver transplant rejection.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

1.-25. (canceled)

26. A method of identifying the status of a liver of a subject after a liver transplant, comprising:
(I) obtaining bile collected from-the liver of the subject after the liver transplant;
(II) isolating one or more of membrane particles, exosomes, exosome-like vesicles, and microvesicles from the bile;
(III) detecting expression of at least one marker of liver condition from each of the following groups of markers:
(a) IL1B, IL6 and IL8,
(b) TNF-alpha, FasL, IFNG, granzyme B, CD16 DEFA3, and PRG2,
(c) IL10, TGF beta, CTLA4, PD-1 and FOXP3, and
(d) IL2, IL4 and GMCSF
by a method comprising:
(i) liberating RNA from the isolated membrane particles, exosomes, exosome-like vesicles, and/or microvesicles;
(ii) contacting the liberated RNA with a reverse transcriptase to generate complementary DNA (cDNA); and
(iii) contacting the cDNA with sense and antisense primers that are specific for each of the markers of liver condition and a DNA polymerase in order to generate amplified DNA; and
(IV) identifying status of the liver of the subject as:
(a) in an early stage of acute rejection or early stage of acute infection when one or more of IL1B, IL6, and IL8 is detected,
(b) in acute rejection when one or more of TNF-alpha, FasL, IFNG, granzyme B, CD16, and DEFA3 is detected,
(c) in a recovery phase from acute rejection when one or more of IL10, TGF-beta, CTLA4, PD-1 and FOXP3 is detected, or
(d) in sustained rejection when one or more of IL2, IL4 and GMCSF is detected.

27.-46. (canceled)


SEQUENCE LISTING (34 sequences)

SEQ ID NO: 1 - DNA, 18 bp, Homo sapiens
  misc_feature: Beta Actin Forward Primer
cctggcacccagcacaat

SEQ ID NO: 2 - DNA, 20 bp, Homo sapiens
  misc_feature: Beta Actin Reverse Primer
gccgatccacacggagtact

SEQ ID NO: 3 - DNA, 20 bp, Homo sapiens
  misc_feature: Albumin Forward Primer
tgcaaggctgacgataagga

SEQ ID NO: 4 - DNA, 26 bp, Homo sapiens
  misc_feature: Albumin Reverse Primer
gtaggctgagatgcttttaaatgtga

SEQ ID NO: 5 - DNA, 21 bp, Homo sapiens
  misc_feature: Human Growth Factor Forward Primer
tccacggaagaggagatgaga

SEQ ID NO: 6 - DNA, 26 bp, Homo sapiens
  misc_feature: Human Growth Factor Reverse Primer
tcattaaaaccagatctgatccttca

SEQ ID NO: 7 - DNA, 20 bp, Homo sapiens
  misc_feature: Vascular Endothelial Growth Factor Forward Primer
cgcagctactgccatccaat

SEQ ID NO: 8 - DNA, 24 bp, Homo sapiens
  misc_feature: Vascular Endothelial Growth Factor
tggcttgaagatgtactcgatctc

SEQ ID NO: 9 - DNA, 24 bp, Homo sapiens
  misc_feature: IL1B Forward Primer
gaagatggaaaagcgatttgtctt

SEQ ID NO: 10 - DNA, 21 bp, Homo sapiens
  misc_feature: IL1B Reverse Primer
gggcatgttttctgcttgaga

SEQ ID NO: 11 - DNA, 27 bp, Homo sapiens
  misc_feature: IL2 Forward Primer
gaactaaagggatctgaaacaacattc

SEQ ID NO: 12 - DNA, 24 bp, Homo sapiens
  misc_feature: IL2 Reverse Primer
tgttgagatgatgctttgacaaaa

SEQ ID NO: 13 - DNA, 20 bp, Homo sapiens
  misc_feature: IL4 Forward Primer
cacaggcacaagcagctgat

SEQ ID NO: 14 - DNA, 23 bp, Homo sapiens
  misc_feature: IL4 Reverse Primer
ccttcacaggacaggaattcaag

SEQ ID NO: 15 - DNA, 24 bp, Homo sapiens
  misc_feature: IL6 Forward Primer
tcatcactggtcttttggagtttg

SEQ ID NO: 16 - DNA, 20 bp, Homo sapiens
  misc_feature: IL6 Reverse Primer
tctgcacagctctggcttgt

SEQ ID NO: 17 - DNA, 27 bp, Homo sapiens
  misc_feature: IL8 Forward Primer
tgctaaagaacttagatgtcagtgcat

SEQ ID NO: 18 - DNA, 23 bp, Homo sapiens
  misc_feature: IL8 Reverse Primer
tggtccactctcaatcactctca

SEQ ID NO: 19 - DNA, 24 bp, Homo sapiens
  misc_feature: IL10 Forward Primer
gccatgagtgagtttgacatcttc

SEQ ID NO: 20 - DNA, 29 bp, Homo sapiens
  misc_feature: IL10 Reverse Primer
gattttggagacctctaatttatgtccta

SEQ ID NO: 21 - DNA, 22 bp, Homo sapiens
  misc_feature: TNFSF2 Forward Primer
cgaaggctccaaagaagacagt

SEQ ID NO: 22 - DNA, 20 bp, Homo sapiens
  misc_feature: TNFSF2 Reverse Primer
cagggcaatgatcccaaagt

SEQ ID NO: 23 - DNA, 24 bp, Homo sapiens
  misc_feature: TNFSF6 Forward Primer
tggcagcatcttcacttctaaatg

SEQ ID NO: 24 - DNA, 24 bp, Homo sapiens
  misc_feature: TNFSF6 Reverse Primer
gaaatgagtccccaaaacatctct

SEQ ID NO: 25 - DNA, 21 bp, Homo sapiens
  misc_feature: DEFA3 Forward Primer
ccaggctcaaggaaaaacatg

SEQ ID NO: 26 - DNA, 23 bp, Homo sapiens
  misc_feature: DEFA3 Reverse Primer
ctggtagatgcaggttccatagc

SEQ ID NO: 27 - DNA, 22 bp, Homo sapiens
  misc_feature: CD16 Forward Primer
gtttggcagtgtcaaccatctc

SEQ ID NO: 28 - DNA, 23 bp, Homo sapiens
  misc_feature: CD16 Reverse Primer
aaaaggagtaccatcaccaagca

SEQ ID NO: 29 - DNA, 18 bp, Homo sapiens
  misc_feature: GMCSF Forward Primer
ggccccttgaccatgatg

SEQ ID NO: 30 - DNA, 21 bp, Homo sapiens
  misc_feature: GMCSF Reverse Primer
tctgggttgcacaggaagttt

SEQ ID NO: 31 - DNA, 23 bp, Homo sapiens
  misc_feature: IFNG Forward Primer
ggagaccatcaaggaagacatga

SEQ ID NO: 32 - DNA, 20 bp, Homo sapiens
  misc_feature: IFNG Reverse Primer
gctttgcgttggacattcaa

SEQ ID NO: 33 - DNA, 19 bp, Homo sapiens
  misc_feature: PRG2 Forward Primer
actgcgtggccctgtgtac

SEQ ID NO: 34 - DNA, 27 bp, Homo sapiens
  misc_feature: PRG2 Reverse Primer
cagtaggaacagatgaaaggaagtctt